SEQUENCE LISTING (42 sequences)

SEQ ID NO: 1 - protein, 10 aa, synthetic construct
GYTFTTYWIQ

SEQ ID NO: 2 - protein, 17 aa, synthetic construct
AVYPGDADTRYTQKFQG

SEQ ID NO: 3 - protein, 11 aa, synthetic construct
DAGLEYYALDY

SEQ ID NO: 4 - protein, 5 aa, synthetic construct
TYWIQ

SEQ ID NO: 5 - protein, 17 aa, synthetic construct
AVYPGDADTRYTQKFQG

SEQ ID NO: 6 - protein, 11 aa, synthetic construct
DAGLEYYALDY

SEQ ID NO: 7 - protein, 7 aa, synthetic construct
GYTFTTY

SEQ ID NO: 8 - protein, 6 aa, synthetic construct
YPGDAD

SEQ ID NO: 9 - protein, 11 aa, synthetic construct
DAGLEYYALDY

SEQ ID NO: 10 - protein, 8 aa, synthetic construct
GYTFTTYW

SEQ ID NO: 11 - protein, 8 aa, synthetic construct
VYPGDADT

SEQ ID NO: 12 - protein, 13 aa, synthetic construct
GRDAGLEYYALDY

SEQ ID NO: 13 - protein, 120 aa, synthetic construct
QVQLVQSGAEVKKPGASVKVSCKASGYTFTTYWIQWVRQAPGQRLEWMGAVYPGDADTRYTQKFQGRVTLTADRSASTAYMELSSLRSEDTAVYYCGRDAGLEYYALDYWGQGTLVTVSS

SEQ ID NO: 14 - protein, 11 aa, synthetic construct
RASQDVGTAVA

SEQ ID NO: 15 - protein, 7 aa, synthetic construct
WASTRHT

SEQ ID NO: 16 - protein, 10 aa, synthetic construct
QQYANFPLYT

SEQ ID NO: 17 - protein, 11 aa, synthetic construct
RASQDVGTAVA

SEQ ID NO: 18 - protein, 7 aa, synthetic construct
WASTRHT

SEQ ID NO: 19 - protein, 10 aa, synthetic construct
QQYANFPLYT

SEQ ID NO: 20 - protein, 7 aa, synthetic construct
SQDVGTA

SEQ ID NO: 21 - skipped

SEQ ID NO: 22 - protein, 7 aa, synthetic construct
YANFPLY

SEQ ID NO: 23 - protein, 6 aa, synthetic construct
QDVGTA

SEQ ID NO: 24 - skipped

SEQ ID NO: 25 - protein, 10 aa, synthetic construct
QQYANFPLYT

SEQ ID NO: 26 - protein, 108 aa, synthetic construct
EIVMTQSPATLSVSPGERATLSCRASQDVGTAVAWYQQKPGQAPRLLIYWASTRHTGIPARFSGSGSGTEFTLTISSLQSEDFAVYFCQQYANFPLYTFGQGTKLEIK

SEQ ID NO: 27 - protein, 5 aa, synthetic construct
TYAMN

SEQ ID NO: 28 - protein, 19 aa, synthetic construct
RIRSKYNNYATYYADSVKD

SEQ ID NO: 29 - protein, 14 aa, synthetic construct
HGNFGNSYVSWFAY

SEQ ID NO: 30 - protein, 14 aa, synthetic construct
RSSTGAVTTSNYAN

SEQ ID NO: 31 - protein, 7 aa, synthetic construct
GTNKRAP

SEQ ID NO: 32 - protein, 9 aa, synthetic construct
ALWYSNLWV

SEQ ID NO: 33 - protein, 125 aa, synthetic construct
EVQLVESGGGLVQPGGSLKLSCAASGFTFNTYAMNWVRQASGKGLEWVGRIRSKYNNYATYYADSVKDRFTISRDDSKSTLYLQMNSLKTEDTAVYYCVRHGNFGNSYVSWFAYWGQGTLVTVSS

SEQ ID NO: 34 - protein, 109 aa, synthetic construct
QAVVTQEPSLTVSPGGTVTLTCRSSTGAVTTSNYANWVQQKPGQAPRGLIGGTNKRAPWTPARFSGSLLGDKAALTLSGAQPEDEAEYFCALWYSNLWVFGGGTKLTVL

SEQ ID NO: 35 - protein, 254 aa, synthetic construct
EVQLVESGGGLVQPGGSLKLSCAASGFTFNTYAMNWVRQASGKGLEWVGRIRSKYNNYATYYADSVKDRFTISRDDSKSTLYLQMNSLKTEDTAVYYCVRHGNFGNSYVSWFAYWGQGTLVTVSSGGGGSGGGGSGGGGSGGGGSQAVVTQEPSLTVSPGGTVTLTCRSSTGAVTTSNYANWVQQKPGQAPRGLIGGTNKRAPWTPARFSGSLLGDKAALTLSGAQPEDEAEYFCALWYSNLWVFGGGTKLTVL

SEQ ID NO: 36 - protein, 94 aa, synthetic construct
SQQIYGVVYGNVTFHVPSNVPLKEVLWKKQKDKVAELENSEFRAFSSFKNRVYLDTVSGSLTIYNLTSSDEDEYEMESPNITDTMKFFLYVLES

SEQ ID NO: 37 - protein, 719 aa, synthetic construct
QVQLVQSGAEVKKPGASVKVSCKASGYTFTTYWIQWVRQAPGQRLEWMGAVYPGDADTRYTQKFQGRVTLTADRSASTAYMELSSLRSEDTAVYYCGRDAGLEYYALDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCGGGGSGGGGSEVQLVESGGGLVQPGGSLKLSCAASGFTFNTYAMNWVRQASGKGLEWVGRIRSKYNNYATYYADSVKDRFTISRDDSKSTLYLQMNSLKTEDTAVYYCVRHGNFGNSYVSWFAYWGQGTLVTVSSGGGGSGGGGSGGGGSGGGGSQAVVTQEPSLTVSPGGTVTLTCRSSTGAVTTSNYANWVQQKPGQAPRGLIGGTNKRAPWTPARFSGSLLGDKAALTLSGAQPEDEAEYFCALWYSNLWVFGGGTKLTVLGGGGSDKTHTCPPCPAPELLGGPSVFLFPPKPKDTLMISRTPEVTCVVVAVSHEDPEVKFNWYVDGVEVHNAKTKPREEQYASTYRVVSVLTVLHQDWLNGKEYKCKVSNKALAAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 38 - protein, 215 aa, synthetic construct
EIVMTQSPATLSVSPGERATLSCRASQDVGTAVAWYQQKPGQAPRLLIYWASTRHTGIPARFSGSGSGTEFTLTISSLQSEDFAVYFCQQYANFPLYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 39 - protein, 326 aa, synthetic construct
SQQIYGVVYGNVTFHVPSNVPLKEVLWKKQKDKVAELENSEFRAFSSFKNRVYLDTVSGSLTIYNLTSSDEDEYEMESPNITDTMKFFLYVLESGGGGSDKTHTCPPCPAPELLGGPSVFLFPPKPKDTLMISRTPEVTCVVVAVSHEDPEVKFNWYVDGVEVHNAKTKPREEQYASTYRVVSVLTVLHQDWLNGKEYKCKVSNKALAAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 40 - DNA, 2205 bp, synthetic construct
  source 1..2205: mol_type = other DNA
atgccactgctgcttctactgccactcctgtgggcaggagcactggcccaagtgcaactggtgcagtccggtgccgaagtgaagaagcccggtgcctctgtgaaggtgtcctgcaaggcgtcgggatacacgttcaccacttactggattcagtgggtcagacaggccccgggacagagactggagtggatgggagccgtgtaccccggagatgcagacactcgctacacccagaagttccagggccgcgtgactttgaccgccgacagaagcgccagcaccgcctacatggagctttcatccctccggagcgaggatactgccgtatactattgcggaagggatgccggcctggaatactatgccctcgactactggggacaggggaccctcgtgactgtgtccagcgcgagcaccaagggccccagcgtgttcccgctggccccatcatccaagtccacctcgggagggactgctgcgctcggttgccttgtgaaggactacttccccgagcccgtgactgtgtcgtggaacagcggggctctgaccagcggggttcacacctttcccgccgtgctgcagtcctcgggactctacagcctgtcctccgtggtcacggtcccgtcgtcgtcgctggggacccagacctacatttgcaacgtgaaccacaaaccctccaacacaaaagtggacaaaagggtggaacctaagtcctgtggagggggtggatcaggcggaggaggatcggaagtccagctcgtcgaatcagggggagggcttgtgcaaccaggaggctccctcaagctgtcttgcgcagcgtccggtttcactttcaacacttatgcgatgaattgggtccgccaagccagtgggaagggcctggagtgggtcggacggatcagatccaagtacaacaactacgcgacatactacgccgactccgtgaaggatcgcttcaccatcagccgggatgactccaagagcaccttgtacctccaaatgaacagccttaagaccgaggacactgcggtgtactactgcgtgagacacggcaacttcggaaactcctacgtgtcctggttcgcctactggggacagggcacccttgtcactgtgtcaagcggaggcggtggttcgggtggcggaggttccggaggaggaggttcgggcggtggtggatcacaggccgtcgtgactcaggaaccatccctgactgtgtcccccggtggaaccgtgaccctcacctgtcgctcctcaaccggagccgtgaccacctccaactacgctaattgggtgcagcagaagccaggacaagccccacggggactgattgggggcaccaacaagagggctccttggaccccagcccgcttctcgggctccctgttgggcgacaaggccgctctgaccctgtccggtgcacagccggaggatgaagccgaatacttctgcgcgctgtggtactccaacctctgggtgttcggcggagggaccaagctgactgtgttgggaggaggggggagtgacaagactcacacgtgtccgccttgcccagcacccgagctactgggaggaccgagcgtgttcctgtttcccccgaagccgaaggataccctgatgatctcccgcactcctgaagtgacttgcgtggtggtggcagtgtcccacgaggacccggaagtcaagtttaattggtacgtggatggcgtggaggtgcacaacgcaaagaccaagcctcgcgaggagcagtacgccagcacctaccgggtggtgtccgtcctgacggtgctgcaccaggactggctgaacgggaaggagtacaagtgcaaagtgtcaaataaggctttggccgcccctattgagaaaaccatctcaaaggccaagggccaacccagggaacctcaagtgtgcaccctcccaccttcgcgagaagagatgaccaagaaccaggtgtccctgtcctgcgccgtgaagggcttctacccctccgatatcgccgtggagtgggaatctaacggacagccggagaacaactacaagaccactccgccggtgctggacagcgacggctccttcttcctcgtgtcgaaactgaccgtggacaagtcacggtggcagcagggcaatgtgttcagctgctcagtcatgcatgaggccctccacaaccactacactcagaagtccctgtcgctttcccccggaaaa

SEQ ID NO: 41 - DNA, 705 bp, synthetic construct
  source 1..705: mol_type = other DNA
atgtcggtcctgacccaagtgctggccctccttctcctgtggctgaccgggaccagatgcgaaatcgtcatgactcagagcccggcaaccctgtccgtgagccctggagaacgggccactctgagctgtcgggcgtcacaggacgtgggaactgccgtggcctggtatcagcagaagccgggacaggctcctaggttgctcatctactgggcgtccactcgccacaccggaatcccagcccgcttctccggctcgggttctggcaccgagttcaccctgaccatttcctccctccaatccgaggatttcgccgtgtacttctgccaacaatacgccaacttccccctgtacacatttggccaggggaccaagctggagattaagcgtacggtggccgctcccagcgtgttcatcttcccccccagcgacgagcagctgaagagcggcaccgccagcgtggtgtgcctgctgaacaacttctacccccgggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcataaggtgtacgcctgcgaggtgacccaccagggcctgtccagccccgtgaccaagagcttcaacaggggcgagtgc

SEQ ID NO: 42 - DNA, 1026 bp, synthetic construct
  source 1..1026: mol_type = other DNA
atgcctctgctgctcctgctgcctctgctctgggccggagctttggcatcacagcaaatctacggcgtggtgtacggcaacgtgaccttccatgtcccctccaatgtgccgctgaaggaagtgctctggaagaagcagaaggacaaggtcgcggaactggaaaactccgagtttcgcgccttctcctccttcaaaaaccgggtgtacctggacaccgtgtccgggagccttactatctacaacctgacctcctcggacgaggatgagtatgagatggagagcccaaacattaccgacaccatgaagttcttcctctacgtgctggaatcgggtggaggcggaagcgataagactcacacgtgtccaccttgtcccgcacccgaactcctggggggaccttccgtgtttctcttcccccctaaaccgaaggacaccttgatgatctcccgcactcctgaagtgacctgtgtggtggtggccgtgtcccacgaggacccagaagtcaagtttaattggtacgtggacggagtcgaggtgcacaacgcgaaaaccaaaccgcgggaggagcagtacgcctccacctaccgggtggtgtccgtcctcactgtgctgcaccaggactggctcaacggaaaggagtacaagtgcaaagtgtccaacaaagccttggcggccccaatcgaaaagacgatctccaaggccaagggacagccgcgcgaacctcaagtctacaccctgcctccttgccgcgaggaaatgaccaagaaccaggtgtcactgtggtgtctggtcaagggattctacccttccgatatcgcagtggagtgggaaagcaacggccaaccagagaacaactataagaccacacccccggtgctcgattccgacggctcattcttcctgtactccaagctgaccgtggacaagtcacggtggcagcaggggaacgtgttcagctgctccgtgatgcatgaagccctgcacaatcattacactcagaagtccctgtcgctgagccccggaaaa

(19) United States
(12) Patent Application Publication
(10) Pub. No.: US 2025/0340634 A1
(43) Pub. Date: Nov. 6, 2025

(54) DOSAGE REGIMENS FOR ANTI-CD19 AGENTS AND USES THEREOF

(71) Applicant: Novartis AG, Basel (CH)

(72) Inventors: Haihui LU, Winchester, MA (US); Adwait OKA, Everett, MA (US); Alessandro PASTORE, Kirschubluetenweg (CH); Joseph Ryan POLLI, Cambridge, MA (US); Janghee WOO, Johns Creek, GA (US)

(21) Appl. No.: 18/856,230

(22) PCT Filed: Apr. 12, 2023
(86) PCT No.: PCT/IB2023/053732

§ 371 Date: Oct. 11, 2024

Related U.S. Application Data
(60) Provisional application No. 63/363,010, filed on Apr. 14, 2022.

(51) Int. Cl. C07K 16/28; A61K 39/00; A61P 35/00; A61P 35/02

(52) U.S. Cl. CPC C07K 16/2803; A61P 35/00; A61P 35/02; C07K 16/2806; C07K 16/2809; C07K 16/2866; A61K 2039/505; A61K 2039/54; A61K 2039/545; C07K 2317/31; C07K 2317/73

(57) ABSTRACT

The present disclosure relates to dosage regimes of anti-CD19 agents, in particular an anti-CD19×anti-CD3×anti-CD2 trispecific agent administered intravenously (i.v.) and subcutaneously (s.c.), and their use for treating diseases and disorders associated with expression of CD19 such as B cell malignancies, in particular relapsed and/or refractory B-cell malignancies.

### 1. FIELD OF INVENTION

The disclosure generally relates to dosage regimes of anti-CD19 agents, in particular an anti-CD19×anti-CD3×anti-CD2 trispecific agent administered intravenously (i.v.) and subcutaneously (s.c.), and their use for treating diseases and disorders associated with expression of CD19 such as B cell malignancies, in particular relapsed and/or refractory B-cell malignancies.

### 2. BACKGROUND

B cells express a wide array of cell surface molecules during their differentiation and proliferation. CD19 is a pan-B cell membrane glycoprotein that is expressed from early stages of pre-B cell development through terminal differentiation, regulating B lymphocyte development and function. Expression of CD19 was identified on the vast majority of Non-Hodgkin lymphoma (NHL) and on leukemias, including Chronic Lymphocytic Leukemia (CLL), Acute Lymphoblastic Leukemia (ALL) and Waldenstrom's Macroglobulinemia (WM).

Relapsed and/or refractory (R/R) NHL patients with treatment failure after initial therapy have a poor outcome. Approximately 50% of patients have a response to initial salvage therapy and then undergo allogenic stem cell transplant (ASCT), with an overall cure rate in the range of 25 to 35%. Patients who are not candidates for ASCT because of poor fitness due to age or co-existing medical conditions, those who do not have a response to salvage therapy, and those who have a relapse after ASCT are classified as transplantation ineligible. Ultimately, most patients with R/R NHL fall into this category, and sequential single agent chemotherapy or a multiagent regimen with an acceptable side effect profile, such as rituximab, gemcitabine and oxaliplatin (R-GemOx) has frequently been used with palliative intent.

Despite treatment advances for R/R ALL, disease recurrence and relapse in patients with increasingly refractory disease continues to be a major obstacle. Treatment options for R/R ALL patients remain limited and include high-dose chemotherapy with subsequent stem-cell transplantation (SCT), standard chemoimmunotherapy, CD19 or CD22 directed therapies (blinatumomab, Inotuzumab ozogamicin), targeted treatment with small molecule pathway inhibitors or supportive care with non-curative palliative goals, but the duration of remission is typically short.

NHLs are sometimes classified into immature lymphoid neoplasms, mature B-cell neoplasms, T-cell and natural killer (NK) cell neoplasms, and post-transplant lymphoproliferative disorders (PTLD). Mature B-cell lymphomas may be further classified into indolent lymphomas (e.g. follicular lymphoma, small lymphocytic lymphoma, marginal-zone lymphoma) and aggressive lymphomas (e.g. diffuse large B-cell lymphoma (DLBCL), mantle cell lymphoma (MCL)).

DLBCL is the most frequent aggressive lymphoma subtype representing 30-35% of all NHL (Ghielmini et al 2013). Approximately one-third of DLBCL patients will develop relapsed and/or refractory (R/R) disease, which represents a major cause of morbidity and mortality. Relapsed and refractory patients have a poor prognosis. In the SCHOLAR-1 study, which combined data from two clinical trials, as well as two academic databases, the median overall survival of 636 patients with refractory DLBCL was only 6.3 months, whereas only 20% of the patients were alive after 2 years (Crump et al 2017).

Follicular lymphoma (FL) is the second most common type of NHL. It is the most common of the clinically indolent NHLs defined as those lymphomas in which survival of the untreated patient is measured in years. The vast majority of patients treated for FL will have an initial response to therapy with 40-80% demonstrating a complete response, depending on the initial regimen used. However, conventional therapy for FL is not curative and most of these patients will ultimately develop progressive disease. There is still a high unmet medical need for patients with R/R FL.

Acute lymphoblastic lymphoma (ALL) is characterized by an accumulation of malignant lymphoblasts in the bone marrow, the peripheral circulation and the lymphoid organs. Blinatumomab, a CD19-CD3 bispecific T cell engager, is approved for the treatment of the treatment of ALL. However, treatment with blinatumomab lacks a durable response and is characterized by a high relapse rate. Von Stackelberg et al., 2016, Journal of Clinical Oncology 34(36):4381-4389. Moreover, blinatumomab has a short half-life, which requires continuous exposure for the drug to exert sufficient efficacy and manageable toxicity. Porter et al., 2013, Clin Pharmacol. 5(Suppl 1): 5-11.

Despite major improvements in cancer therapy, B cell malignancies, such as the B cell subtypes of non-Hodgkin's lymphomas are major contributors of cancer-related deaths. In particular, patients with R/R B-NHL and R/R B-ALL who have failed multiple prior lines of therapy have a high unmet medical need. There is still a high unmet medical need for patients with R/R DLBCL who are not eligible for or do not have access to autologous stem cell transplant (ASCT) and CAR-T therapy. These patients have few effective treatment options and significantly reduced overall survival (OS). There is also a high unmet need for patients who relapse after transplant or CAR-T therapy. Accordingly, there is still a need for further therapeutic agents for the treatment of certain B cell malignancies.

### 3. SUMMARY

In some embodiments the invention relates to a method of treating a subject having a B-cell malignancy by administering an anti-CD19×anti-CD3×anti-CD2 trispecific agent. In some embodiments the invention relates to an anti-CD19×anti-CD3×anti-CD2 trispecific agent for use in treating a B-cell malignancy. The B-cell malignancy may be a relapsed and/or refractory B-cell malignancy. The B-cell malignancy may be selected from the group consisting of R/R LBCL, R/R DLBCL, R/R HGBCL, R/R PMBCL, R/R FL, R/R FL3B, R/R MCL, R/R SLL, R/R MZL and R/R ALL.

In some embodiments the invention relates to a method of treating a subject with a CD19 associated disease or disorder by administering an anti-CD19×anti-CD3×anti-CD2 trispecific agent. In some embodiments the invention relates to an anti-CD19×anti-CD3×anti-CD2 trispecific agent for use in treating a CD19 associated disease or disorder. The CD19 associated disease or disorder may be a relapsed and/or refractory disease or disorder. The CD19 associated disease or disorder may be selected from the group consisting of R/R LBCL, R/R DLBCL, R/R HGBCL, R/R PMBCL, R/R FL, R/R FL3B, R/R MCL, R/R SLL, R/R MZL and R/R ALL. In some embodiments, the CD19 associated disease or disorder is systemic lupus erythematosus (SLE).

In some embodiments, the anti-CD19×anti-CD3×anti-CD2 trispecific agent can comprise a CD19 binding portion with a CDR-H1, a CDR-H2, and a CDR-H3 having the amino acid sequences of SEQ ID NO:4, SEQ ID NO:5, and SEQ ID NO:6, and a CDR-L1, a CDR-L2, and a CDR-L3 having the amino acid sequences of SEQ ID NO:17, SEQ ID NO:18, and SEQ ID NO:19.

In some embodiments, the anti-CD19×anti-CD3×anti-CD2 trispecific agent can comprise (a) a first polypeptide whose amino acid sequence comprises the amino acid sequence of SEQ ID NO:37; (b) a second polypeptide whose amino acid sequence comprises the amino acid sequence of SEQ ID NO:38; and (c) a third polypeptide whose amino acid sequence comprises the amino acid sequence of SEQ ID NO:39. In some embodiments, the anti-CD19×anti-CD3×anti-CD2 trispecific agent comprises the amino acid sequences given in Table 5.

In some embodiments, the anti-CD19×anti-CD3×anti-CD2 trispecific agent can comprise (a) a first polypeptide encoded by a nucleotide sequence which comprises the sequence of SEQ ID NO:40; (b) a second polypeptide encoded by a nucleotide sequence which comprises the sequence of SEQ ID NO:41; and (c) a third polypeptide encoded by a nucleotide sequence which comprises the sequence of SEQ ID NO:42. In some embodiments, the anti-CD19×anti-CD3×anti-CD2 trispecific agent is encoded by any of the nucleotide sequences given in Table 6.

In some embodiments, the anti-CD19×anti-CD3×anti-CD2 trispecific agent comprises any of the sequences given in Tables 1-6. In some embodiments, the anti-CD19×anti-CD3×anti-CD2 trispecific agent is CD19TSP1.

In some embodiments the invention relates to a method of treating a subject having a B-cell malignancy by administering a trispecific antibody. In some embodiments the invention relates to a trispecific antibody for use in treating a B-cell malignancy. The B-cell malignancy may be a relapsed and/or refractory B-cell malignancy. In some embodiments, the trispecific antibody can comprise (a) a first polypeptide whose amino acid sequence comprises the amino acid sequence of SEQ ID NO:37; (b) a second polypeptide whose amino acid sequence comprises the amino acid sequence of SEQ ID NO:38; and (c) a third polypeptide whose amino acid sequence comprises the amino acid sequence of SEQ ID NO:39. In some embodiments, the trispecific antibody comprises the amino acid sequences given in Table 5. The B-cell malignancy may selected from the group consisting of LBCL, DLBCL, HGBCL, PMBCL, FL, FL3B, MCL, SLL, MZL and ALL. The B-cell malignancy may selected from the group consisting of R/R LBCL, R/R DLBCL, R/R HGBCL, R/R PMBCL, R/R FL, R/R FL3B, R/R MCL, R/R SLL, R/R MZL and R/R ALL.

In some embodiments the invention relates to a method of treating a subject having a condition selected from LBCL, DLBCL, HGBCL, PMBCL, FL, FL3B, MCL, SLL, MZL and ALL comprising administering to the subject a therapeutically effective amount of an anti-CD19×anti-CD3×anti-CD2 trispecific agent. In some embodiments the invention relates to an anti-CD19×anti-CD3×anti-CD2 trispecific agent for use in treating a condition selected from LBCL, DLBCL, HGBCL, PMBCL, FL, FL3B, MCL, SLL, MZL and ALL. In some embodiments the invention relates to an anti-CD19×anti-CD3×anti-CD2 trispecific agent for use in treating a condition selected from LBCL, DLBCL, HGBCL, PMBCL, FL, FL3B, MCL, SLL, MZL, ALL and SLE. In some embodiments the condition is LBCL or FL3B, optionally relapsed and/or refractory LBCL or FL3B. In some embodiments the condition is an autoimmune condition, optionally systemic lupus erythematosus (SLE).

In some embodiments the invention relates to a method of treating a subject having a Non-Hodgkin Lymphoma (NHL) by administering an anti-CD19×anti-CD3×anti-CD2 trispecific agent to a subject in need thereof. The NHL may be relapsed and/or refractory NHL. The NHL may be relapsed and/or refractory B-cell Non-Hodgkin Lymphoma (R/R B-NHL). In some embodiments the invention relates to a method of treating a subject having a large B-cell lymphoma (LBCL) by administering an anti-CD19×anti-CD3×anti-CD2 trispecific agent to a subject in need thereof. The LBCL may be relapsed and/or refractory LBCL. In some embodiments the invention relates to a method of treating a subject having a Diffuse Large B-cell Lymphoma (DLBCL) by administering an anti-CD19×anti-CD3×anti-CD2 trispecific agent to a subject in need thereof. The DLBCL may be relapsed and/or refractory DLBCL. The DLBCL may be de novo or transformed. In some embodiments the invention relates to a method of treating a subject having High-grade B-cell lymphoma by administering an anti-CD19×anti-CD3×anti-CD2 trispecific agent to a subject in need thereof. The lymphoma may be double/triple hit High-grade B-cell lymphoma (HGBCL). The HGBCL may be relapsed and/or refractory HGBCL. The HGBCL may be relapsed and/or refractory double/triple hit HGBCL. In some embodiments the invention relates to a method of treating a subject having Primary mediastinal large B-cell lymphoma (PMBCL) by administering an anti-CD19×anti-CD3×anti-CD2 trispecific agent to a subject in need thereof. The PMBCL may be relapsed and/or refractory PMBCL. In some embodiments the invention relates to a method of treating a subject having a Follicular lymphoma (FL) by administering an anti-CD19×anti-CD3×anti-CD2 trispecific agent to a subject in need thereof. The FL may be relapsed and/or refractory FL. In some embodiments the invention relates to a method of treating a subject having a Follicular lymphoma grade 3B (FL3B) by administering an anti-CD19×anti-CD3×anti-CD2 trispecific agent to a subject in need thereof. The FL3B may be relapsed and/or refractory FL3B. In some embodiments the invention relates to a method of treating a subject having mantle cell lymphoma (MCL) by administering an anti-CD19×anti-CD3×anti-CD2 trispecific agent to a subject in need thereof. The MCL may be relapsed and/or refractory MCL. In some embodiments the invention relates to a method of treating a subject having small lymphocytic lymphoma (SLL) by administering an anti-CD19×anti-CD3×anti-CD2 trispecific agent to a subject in need thereof. The SLL may be relapsed and/or refractory SLL. In some embodiments the invention relates to a method of treating a subject having marginal zone lymphoma (MZL) (e.g. nodal, extranodal or mucosa associated) by administering an anti-CD19×anti-CD3×anti-CD2 trispecific agent to a subject in need thereof. The MZL may be relapsed and/or refractory MZL. Optionally, in each case above the treatment may be after previous CAR-T therapy, e.g. CD19 directed CAR-T therapy. In an alternative option, the treatment may be without previous CAR-T therapy, e.g. CD19 directed CAR-T therapy. Optionally in each case above the treatment may be after treatment with a CD20 monoclonal antibody containing chemotherapy regimen. Furthermore, optionally in each case above the treatment may be after prior autologous hematopoietic stem cell transplantation (HSCT). The Eastern Cooperative Oncology Group (ECOG) performance status of the subject may be less than or equal to two.

In some embodiments the invention relates to an anti-CD19×anti-CD3×anti-CD2 trispecific agent for use in treating Non-Hodgkin Lymphoma (NHL). The NHL lymphoma may be relapsed and/or refractory NHL. The NHL may be relapsed and/or refractory B-cell Non-Hodgkin Lymphoma (R/R B-NHL). In some embodiments the invention relates to an anti-CD19×anti-CD3×anti-CD2 trispecific agent for use in treating large B-cell lymphoma (LBCL). The LBCL may be relapsed and/or refractory LBCL. In some embodiments the invention relates to an anti-CD19×anti-CD3×anti-CD2 trispecific agent for use in treating Diffuse Large B-cell Lymphoma (DLBCL). The DLBCL may be relapsed and/or refractory DLBCL. The DLBCL may be de novo or transformed. In some embodiments the invention relates to an anti-CD19×anti-CD3×anti-CD2 trispecific agent for use in treating high-grade B-cell lymphoma (HGBCL). The lymphoma may be double/triple hit High-grade B-cell lymphoma (HGBCL). The HGBCL may be relapsed and/or refractory HGBCL. The HGBCL may be relapsed and/or refractory double/triple hit HGBCL. In some embodiments the invention relates to an anti-CD19×anti-CD3×anti-CD2 trispecific agent for use in treating Primary mediastinal large B-cell lymphoma (PMBCL). The PMBCL may be relapsed and/or refractory PMBCL. In some embodiments the invention relates to an anti-CD19×anti-CD3×anti-CD2 trispecific agent for use in treating Follicular lymphoma. The FL may be relapsed and/or refractory FL. In some embodiments the invention relates to an anti-CD19×anti-CD3×anti-CD2 trispecific agent for use in treating Follicular lymphoma grade 3B (FL3B). The FL3B may be relapsed and/or refractory FL3B. In some embodiments the invention relates to an anti-CD19×anti-CD3×anti-CD2 trispecific agent for use in treating mantle cell lymphoma (MCL). The MCL may be relapsed and/or refractory MCL. In some embodiments the invention relates to an anti-CD19×anti-CD3×anti-CD2 trispecific agent for use in treating small lymphocytic lymphoma (SLL). The SLL may be relapsed and/or refractory SLL. In some embodiments the invention relates to an anti-CD19×anti-CD3×anti-CD2 trispecific agent for use in treating marginal zone lymphoma (MZL) (e.g. nodal, extranodal or mucosa associated). The MZL may be relapsed and/or refractory MZL. Optionally, in each case above the treatment may be after previous CAR-T therapy, e.g. CD19 directed CAR-T therapy. In an alternative option, the treatment may be without previous CAR-T therapy, e.g. CD19 directed CAR-T therapy. Optionally in each case above the treatment may be after treatment with a CD20 monoclonal antibody containing chemotherapy regimen. Furthermore, optionally in each case above the treatment may be after prior autologous hematopoietic stem cell transplantation (HSCT). The treatment may optionally be used when the Eastern Cooperative Oncology Group (ECOG) performance status of the subject may be less than or equal to two.

In some embodiments the invention relates to a method of treating a subject having a relapsed and/or refractory NHL (e.g. DLBCL, HGBCL, PMBCL, FL, FL3B, MCL, SLL, MZL) by administering an anti-CD19×anti-CD3×anti-CD2 trispecific agent to a subject in need thereof, wherein relapsed and/or refractory NHL may be relapsed after or failure to respond to at least two prior treatment regimens including an anti-CD20 monoclonal antibody containing chemotherapy regimen. In some embodiments the invention relates to an anti-CD19×anti-CD3×anti-CD2 trispecific agent for use in treating a relapsed and/or refractory NHL (e.g. DLBCL, HGBCL, PMBCL, FL, FL3B, MCL, SLL, MZL), wherein relapsed and/or refractory may be relapsed after or failure to respond to at least two prior treatment regimens including an anti-CD20 monoclonal antibody containing chemotherapy regimen.

In some embodiments the invention relates to a method of treating a subject having relapsed and/or refractory large B cell lymphoma (DLBCL, HGBCL, PMBCL, FL3B), wherein the relapsed and/or refractory may include failed prior autologous hematopoietic stem cell transplantation (HSCT). In some embodiments the invention relates to a method of treating a subject having relapsed and/or refractory large B cell lymphoma (DLBCL, HGBCL, PMBCL, FL3B), wherein the subject is ineligible for or not able to receive autologous stem cell transplantation due to age and/or comorbidities. In some embodiments the invention relates to an anti-CD19×anti-CD3×anti-CD2 trispecific agent for use in treating relapsed and/or refractory large B cell lymphoma (DLBCL, HGBCL, PMBCL, FL3B), wherein the relapsed and/or refractory may include failed prior autologous hematopoietic stem cell transplantation (HSCT). In some embodiments the invention relates to an anti-CD19×anti-CD3×anti-CD2 trispecific agent for use in treating relapsed and/or refractory large B cell lymphoma (DLBCL, HGBCL, PMBCL, FL3B), wherein the subject is ineligible for or not able to receive autologous stem cell transplantation due to age and/or comorbidities.

In some embodiments the invention relates to a method of treating a subject having NHL (e.g. DLBCL, HGBCL, PMBCL, FL, FL3B, MCL, SLL, MZL) by administering an anti-CD19×anti-CD3×anti-CD2 trispecific agent to a subject in need thereof, wherein the subject may have at least one bi-dimensionally measurable nodal lesion or one bi-dimensionally measurable extranodal lesion, as measured on positron emission tomography-computed tomography (PET/CT) scan. In some embodiments the invention relates to an anti-CD19×anti-CD3×anti-CD2 trispecific agent for use in treating NHL (e.g. DLBCL, HGBCL, PMBCL, FL, FL3B, MCL, SLL, MZL), wherein the subject may have at least one bi-dimensionally measurable nodal lesion or one bi-dimensionally measurable extranodal lesion, as measured on positron emission tomography-computed tomography (PET/CT) scan.

In some embodiments the invention relates to a method of treating a subject having Acute Lymphoblastic Leukemia (ALL) by administering a therapeutically effective amount of anti-CD19×anti-CD3×anti-CD2 trispecific agent to a subject in need thereof. The ALL may be relapsed and/or refractory ALL. The ALL may be relapsed and/or refractory B-cell Acute Lymphoblastic Leukemia (R/R B-ALL). The ALL may be relapsed and/or refractory CD19-positive B-ALL. Optionally, the treatment may be after previous CD19-directed CAR-T therapy. In an alternative option, the treatment may be without previous CD19-directed CAR-T therapy. In some embodiments the invention relates to an anti-CD19×anti-CD3×anti-CD2 trispecific agent for use in treating Acute Lymphoblastic Leukemia (ALL). The ALL may be relapsed and/or refractory ALL. The ALL may be relapsed and/or refractory B-cell Acute Lymphoblastic Leukemia (R/R B-ALL). The ALL may be relapsed and/or refractory CD19-positive B-ALL. Optionally, treatment may be after previous CD19-directed CAR-T therapy. In an alternative option, the treatment may be without previous CD19-directed CAR-T therapy.

In some embodiments the invention relates to a method of treating a subject having an Acute Lymphoblastic Leukemia (ALL) by administering an anti-CD19×anti-CD3×anti-CD2 trispecific agent to a subject in need thereof, wherein morphologic disease is present in the bone marrow (5% blasts). In some embodiments the invention relates to an anti-CD19×anti-CD3×anti-CD2 trispecific agent for use in treating Acute Lymphoblastic Leukemia (ALL), wherein morphologic disease is present in the bone marrow (5% blasts).

In some embodiments the invention relates to a method of treating a subject having Refractory and/or Relapsed CD19-positive B-ALL by administering an anti-CD19×anti-CD3×anti-CD2 trispecific agent to a subject in need thereof. In some embodiments the invention relates to an anti-CD19×anti-CD3×anti-CD2 trispecific agent for use in treating Refractory and/or Relapsed CD19-positive B-ALL. Refractory and/or Relapsed CD19-positive B-ALL may include at least one of the following criteria:
i) After at least 2 or more lines of systemic therapy
ii) Relapsed or Refractory at any time after first salvage therapy or refractory relapse
iii) Relapse at any time after hematopoietic stem cell transplant (HSCT)
iv) Refractory to, or intolerant to, or ineligible/unable to receive SOC therapeutic options including blinatumomab and inotuzumab
v) Patients with R/R B-ALL Ph+ disease and that are intolerant or refractory to available tyrosine kinase inhibitors (TKIs)

The anti-CD19×anti-CD3×anti-CD2 trispecific agent may be administered Q1W or Q2W. The anti-CD19×anti-CD3×anti-CD2 trispecific agent may be administered intravenously or subcutaneously. The anti-CD19×anti-CD3×anti-CD2 trispecific agent may be administered via an initial priming dose, followed by a main dose. The anti-CD19×anti-CD3×anti-CD2 trispecific agent may be administered as a lyophilisate.

The anti-CD19×anti-CD3×anti-CD2 trispecific agent may be administered at 0.1 μg/kg (100 ng/kg) administered Q1W, optionally intravenously. The anti-CD19×anti-CD3×anti-CD2 trispecific agent may be administered at a dose selected from the group consisting of 0.1, 0.3, 1, 3, 10, 20, 40, 80, 160, 320, 640, 1280, 2560 micrograms/kilogram (μg/kg). The dosages are based on the subject's weight measurement in kg. The anti-CD19×anti-CD3×anti-CD2 trispecific agent may be administered via an initial priming dose, e.g. 80 μg/kg followed by a main dose of 160 μg/kg.

In some embodiments the anti-CD19×anti-CD3×anti-CD2 trispecific agent may be administered with one or more of the agents selected from the group consisting of tocilizumab, siltuximab, cyclophosphamide, anti-thymocyte globulin (ATG), alemtuzumab and anakinra. In some embodiments the anti-CD19×anti-CD3×anti-CD2 trispecific agent may be administered with one or more of the agents selected from the group consisting of steroids, anti-IL-6, anti-TNF and anti-IL-1R antibodies. In some embodiments the anti-CD19×anti-CD3×anti-CD2 trispecific agent may be administered with one or more of the agents selected from the group consisting of tocilizumab, siltuximab, cyclophosphamide, anti-thymocyte globulin (ATG), alemtuzumab, anakinra, steroids, anti-IL-6, anti-TNF and anti-IL-1R antibodies. In some embodiments the anti-CD19×anti-CD3×anti-CD2 trispecific agent may be administered with one or more of the agents selected from the group consisting of an antihistamine, steroids (including corticosteroids), or other anti-T cell directed therapy (e.g. tocilizumab or canakinumab). In some embodiments the anti-CD19×anti-CD3×anti-CD2 trispecific agent may be administered with tocilizumab and/or a corticosteriod. For example, the steroid may be prednisone or dexamethasone. In some embodiments the anti-CD19×anti-CD3×anti-CD2 trispecific agent may be administered with a CRS therapy.

In some embodiments the invention relates to an anti-CD19×anti-CD3×anti-CD2 trispecific agent for use in treating a subject having a condition selected from the group consisting of LBCL, DLBCL, HGBCL, PMBCL, FL, FL3B, MCL, SLL, MZL and ALL, wherein the anti-CD19×anti-CD3×anti-CD2 trispecific agent can comprise (a) a first polypeptide whose amino acid sequence comprises the amino acid sequence of SEQ ID NO:37; (b) a second polypeptide whose amino acid sequence comprises the amino acid sequence of SEQ ID NO:38; and (c) a third polypeptide whose amino acid sequence comprises the amino acid sequence of SEQ ID NO:39. In some embodiments the invention relates to an anti-CD19×anti-CD3×anti-CD2 trispecific agent for use in treating a subject having a condition selected from the group consisting of R/R LBCL, R/R DLBCL, R/R HGBCL, R/R PMBCL, R/R FL, R/R FL3B, R/R MCL, R/R SLL, R/R MZL and R/R ALL, wherein the anti-CD19×anti-CD3×anti-CD2 trispecific agent can comprise (a) a first polypeptide whose amino acid sequence comprises the amino acid sequence of SEQ ID NO:37; (b) a second polypeptide whose amino acid sequence comprises the amino acid sequence of SEQ ID NO:38; and (c) a third polypeptide whose amino acid sequence comprises the amino acid sequence of SEQ ID NO:39. In some embodiments the invention relates to an anti-CD19×anti-CD3×anti-CD2 trispecific agent for use in treating a subject having R/R NHL, wherein the anti-CD19×anti-CD3×anti-CD2 trispecific agent can comprise (a) a first polypeptide whose amino acid sequence comprises the amino acid sequence of SEQ ID NO:37; (b) a second polypeptide whose amino acid sequence comprises the amino acid sequence of SEQ ID NO:38; and (c) a third polypeptide whose amino acid sequence comprises the amino acid sequence of SEQ ID NO:39. In some embodiments the invention relates to an anti-CD19×anti-CD3×anti-CD2 trispecific agent for use in treating a subject having R/R ALL, wherein the anti-CD19×anti-CD3×anti-CD2 trispecific agent can comprise (a) a first polypeptide whose amino acid sequence comprises the amino acid sequence of SEQ ID NO:37; (b) a second polypeptide whose amino acid sequence comprises the amino acid sequence of SEQ ID NO:38; and (c) a third polypeptide whose amino acid sequence comprises the amino acid sequence of SEQ ID NO:39. The anti-CD19×anti-CD3×anti-CD2 trispecific agent may be administered Q1W or Q2W. The anti-CD19×anti-CD3×anti-CD2 trispecific agent may be administered intravenously or subcutaneously. The anti-CD19×anti-CD3×anti-CD2 trispecific agent may be administered via an initial priming dose, followed by a main dose.

In some embodiments the invention relates to a method of treating a subject with a CD19 associated disease or disorder which comprises administering an anti-CD19×anti-CD3×anti-CD2 trispecific agent at a dose selected from the group consisting of 0.1, 0.3, 1, 3, 10, 20, 40, 80, 160, 320, 640, 1280, 2560 micrograms/kilogram (μg/kg).

In some embodiments the invention relates to an anti-CD19×anti-CD3×anti-CD2 trispecific agent for use as a medicament, wherein the anti-CD19×anti-CD3×anti-CD2 trispecific agent is administered at a dose selected from the group consisting of 0.1, 0.3, 1, 3, 10, 20, 40, 80, 160, 320, 640, 1280, 2560 micrograms/kilogram (μg/kg).

In some embodiments the anti-CD19×anti-CD3×anti-CD2 trispecific agent can comprise a CD19 binding portion with a CDR-H1, a CDR-H2, and a CDR-H3 having the amino acid sequences of SEQ ID NO:4, SEQ ID NO:5, and SEQ ID NO:6, and a CDR-L1, a CDR-L2, and a CDR-L3 having the amino acid sequences of SEQ ID NO:17, SEQ ID NO:18, and SEQ ID NO:19.

In some embodiments the anti-CD19×anti-CD3×anti-CD2 trispecific agent comprises (a) a first polypeptide whose amino acid sequence comprises the amino acid sequence of SEQ ID NO:37; (b) a second polypeptide whose amino acid sequence comprises the amino acid sequence of SEQ ID NO:38; and (c) a third polypeptide whose amino acid sequence comprises the amino acid sequence of SEQ ID NO:39.

### 3. BRIEF DESCRIPTION OF THE FIGURES

FIG. 1 Diagram showing format of an anti-CD19×anti-CD3×anti-CD2 trispecific agent.

The anti-CD2 portion is shown as a CD58 moeity.

FIG. 2 CD19TSP1 mediates dose dependent killing of Karpas-422 cells

FIG. 3 CD19TSP1 induces dose dependent T cell proliferation in a Karpas-422 and T cell co-culture system

FIG. 4. CD19TSP1 treatment results in secretion of IFNγ, IL-2 and TNFα from Karpas422-T cell co-culture.

FIG. 5. CD19TSP1 showed dose dependent anti-tumor activity in the AdT model with DLBCL xenograft FIG. 6. CD19TSP1 induced sustained decreases in peripheral blood B cell counts in cynomolgus monkeys

### 4. DETAILED DESCRIPTION

As used herein, the following terms are intended to have the following meanings:

Antibody: The term “antibody” as used herein refers to a polypeptide (or set of polypeptides) of the immunoglobulin family that is capable of binding an antigen non-covalently, reversibly and specifically. For example, a naturally occurring “antibody” of the IgG type is a tetramer comprising at least two heavy (H) chains and two light (L) chains inter-connected by disulfide bonds. Each heavy chain is comprised of a heavy chain variable region (abbreviated herein as VH) and a heavy chain constant region. The heavy chain constant region is comprised of three domains, CH1, CH2 and CH3. Each light chain is comprised of a light chain variable region (abbreviated herein as VL) and a light chain constant region. The light chain constant region is comprised of one domain (abbreviated herein as CL). The VH and VL regions can be further subdivided into regions of hypervariability, termed complementarity determining regions (CDR), interspersed with regions that are more conserved, termed framework regions (FR). Each VH and VL is composed of three CDRs and four FRs arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies can mediate the binding of the immunoglobulin to host tissues or factors, including various cells of the immune system (e.g., effector cells) and the first component (Clq) of the classical complement system. The term “antibody” includes, but is not limited to, monoclonal antibodies, human antibodies, humanized antibodies, camelised antibodies, chimeric antibodies, bispecific or multispecific antibodies and anti-idiotypic (anti-Id) antibodies (including, e.g., anti-Id antibodies to antibodies of the disclosure). The antibodies can be of any isotype/class (e.g., IgG, IgE, IgM, IgD, IgA and IgY) or subclass (e.g., IgG1, IgG2, IgG3, IgG4, IgA1 and IgA2).

Both the light and heavy chains are divided into regions of structural and functional homology. The terms “constant” and “variable” are used functionally. In this regard, it will be appreciated that the variable domains of both the light (VL) and heavy (VH) chain portions determine antigen recognition and specificity. Conversely, the constant domains of the light chain (CL) and the heavy chain (CH1, CH2 or CH3) confer important biological properties such as secretion, transplacental mobility, Fc receptor binding, complement binding, and the like. By convention the numbering of the constant region domains increases as they become more distal from the antigen-binding site or amino-terminus of the antibody. In a wild-type antibody, at the N-terminus is a variable region and at the C-terminus is a constant region; the CH3 and CL domains actually comprise the carboxy-terminus of the heavy and light chain, respectively.

Antibody fragment: The term “antibody fragment” of an antibody as used herein refers to one or more portions of an antibody. In some embodiments, these portions are part of the contact domain(s) of an antibody. In some other embodiments, these portion(s) are antigen-binding fragments that retain the ability of binding an antigen non-covalently, reversibly and specifically, sometimes referred to herein as the “antigen-binding fragment”, “antigen-binding fragment thereof,” “antigen-binding portion”, and the like. Examples of binding fragments include, but are not limited to, single-chain Fvs (scFv), a Fab fragment, a monovalent fragment consisting of the VL, VH, CL and CH1 domains; a F(ab)2 fragment, a bivalent fragment comprising two Fab fragments linked by a disulfide bridge at the hinge region; a Fd fragment consisting of the VH and CH1 domains; a Fv fragment consisting of the VL and VH domains of a single arm of an antibody; a dAb fragment (Ward et al., 1989, Nature 341:544-546), which consists of a VH domain; and an isolated complementarity determining region (CDR). Thus, the term “antibody fragment” encompasses both proteolytic fragments of antibodies (e.g., Fab and F(ab)2 fragments) and engineered proteins comprising one or more portions of an antibody (e.g., an scFv).

Antibody fragments can also be incorporated into single domain antibodies, maxibodies, minibodies, intrabodies, diabodies, triabodies, tetrabodies, v-NAR and bis-scFv (see, e.g., Hollinger and Hudson, 2005, Nature Biotechnology 23: 1126-1136). Antibody fragments can be grafted into scaffolds based on polypeptides such as Fibronectin type Ill (Fn3) (see U.S. Pat. No. 6,703,199, which describes fibronectin polypeptide monobodies).

Antibody fragments can be incorporated into single chain molecules comprising a pair of tandem Fv segments (for example, VH-CH1-VH-CH1) which, together with complementary light chain polypeptides (for example, VL-VC-VL-VC), form a pair of antigen-binding regions (Zapata et al., 1995, Protein Eng. 8:1057-1062; and U.S. Pat. No. 5,641,870).

Antibody Numbering System: In the present specification, the references to numbered amino acid residues in antibody domains are based on the EU numbering system unless otherwise specified (for example, in Table 1). This system was originally devised by Edelman et al., 1969, Proc. Nat'l Acad. Sci. USA 63:78-85 and is described in detail in Kabat et al., 1991, in Sequences of Proteins of Immunological Interest, US Department of Health and Human Services, NIH, USA.

The term “anti-CD19×anti-CD3×anti-CD2 trispecific agent” refers to an agent (e.g., a therapeutic agent) targeting CD19, CD3 and CD2.

B cell malignancy: As used herein, a B cell malignancy refers to an uncontrolled proliferation of B cells. Examples of B cell malignancy include non-Hodgkin's lymphomas (NHL), Hodgkin's lymphomas, leukemia, and myeloma. For example, a B cell malignancy can be, but is not limited to, multiple myeloma, chronic lymphocytic leukemia (CLL)/small lymphocytic lymphoma (SLL), large B cell lymphoma (LBCL), follicular lymphoma (FL), Follicular lymphoma grade 3B (FL3B), mantle cell lymphoma (MCL), diffuse large B-cell lymphoma (DLBCL), High-grade B-cell lymphoma (HGBCL), Primary mediastinal large B-cell lymphoma (PMBCL), marginal zone lymphomas (MZL), Burkitt lymphoma, lymphoplasmacytic lymphoma (Waldenstrom macroglobulinemia), hairy cell leukemia, splenic marginal zone B-cell lymphoma, extranodal marginal zone lymphoma (EMZL), nodal marginal zone B-cell lymphoma (NZML), and primary effusion lymphoma.

Cancer: The term “cancer” refers to a disease characterized by the uncontrolled (and often rapid) growth of aberrant cells. Cancer cells can spread locally or through the bloodstream and lymphatic system to other parts of the body. Examples of cancers include the B cell malignancies described herein. The term “cancerous B cell” refers to a B cell that is undergoing or has undergone uncontrolled proliferation.

CD2: The term “CD2” refers to the cluster of differentiation 2 molecule. It may be found on T cells or Natural Killer cells. CD2 interacts with lymphocyte function-associated antigen CD58 (LFA-3) and CD48/BCM1 to mediate adhesion between T-cells and other cell types. CD2 is implicated in the triggering of T-cells, the cytoplasmic domain is implicated in the signaling function. The human and murine amino acid and nucleic acid sequences can be found in a public database, such as GenBank, UniProt and Swiss-Prot. For example, the amino acid sequence of human CD2 can be found as UniProt/Swiss-Prot Accession No. P06729 and the nucleotide sequence encoding of the human CD2 can be found at Accession No. NM_CD001767.5.

CD3: The term “CD3” or “cluster of differentiation 3” refers to the cluster of differentiation 3 co-receptor of the T cell receptor. CD3 helps in activation of both cytotoxic T-cell (e.g., CD8+ naïve T cells) and T helper cells (e.g., CD4+ naïve T cells) and is composed of four distinct chains: one CD3γ chain (e.g., Genbank Accession Numbers NM_CD000073 and MP_CD000064 (human)), one CD36 chain (e.g., Genbank Accession Numbers NM_CD000732, NM_CD001040651, NP_CD00732 and NP_CD001035741 (human)), and two CD3E chains (e.g., Genbank Accession Numbers NM_CD000733 and NP_CD00724 (human)). The chains of CD3 are highly related cell-surface proteins of the immunoglobulin superfamily containing a single extracellular immunoglobulin domain. The CD3 molecule associates with the T-cell receptor (TCR) and ζ-chain to form the T-cell receptor (TCR) complex, which functions in generating activation signals in T lymphocytes. Unless expressly indicated otherwise, the reference to CD3 in the application can refer to the CD3 co-receptor, the CD3 co-receptor complex, or any polypeptide chain of the CD3 co-receptor complex.

CD19: The term “CD19” or “cluster of differentiation 19” refers to the Cluster of Differentiation 19 protein, which is an antigenic determinant detectable on leukemia precursor cells. The human and murine amino acid and nucleic acid sequences can be found in a public database, such as GenBank, UniProt and Swiss-Prot. For example, the amino acid sequence of human CD19 can be found as UniProt/Swiss-Prot Accession No. P15391 and the nucleotide sequence encoding of the human CD19 can be found at Accession No. NM_CD001178098. CD19 is expressed on most B lineage cancers, including, e.g., acute lymphoblastic leukaemia, chronic lymphocytic leukemia (CLL)/small lymphocytic lymphoma (SLL) and non-Hodgkin's lymphoma. It is also an early marker of B cell progenitors. See, e.g., Nicholson et al., 1997, Mol. Immun. 34 (16-17): 1157-1165.

Chimeric Antigen Receptor: The term “Chimeric Antigen Receptor” or alternatively a “CAR” refers to a set of polypeptides, typically two in the simplest embodiments, which when in an immune effector cell, provides the cell with specificity for a target cell, typically a cancer cell, and with intracellular signal generation. In some embodiments, a CAR comprises at least an extracellular antigen binding domain, a transmembrane domain and a cytoplasmic signaling domain (also referred to herein as “an intracellular signaling domain”) comprising a functional signaling domain derived from a stimulatory molecule and/or costimulatory molecule as defined below. The set of polypeptides can be contiguous or non-contiguous with each other. Where the polypeptides are not contiguous with one another, the set of polypeptides include a dimerization switch that, upon the presence of a dimerization molecule, can couple the polypeptides to one another, e.g., can couple an antigen binding domain to an intracellular signaling domain. CAR molecules are typically administered to a subject by way of administration of immune effector cells (e.g., T cells that are preferably autologous to the subject) engineered to express a CAR molecule.

In Combination: Administered “in combination,” as used herein, means that two (or more) different treatments are delivered to the subject during the course of the subject's affliction with the disorder, e.g., the two or more treatments are delivered after the subject has been diagnosed with the disorder and before the disorder has been cured or eliminated or treatment has ceased for other reasons. The terms “combination” or “in combination with” are not intended to imply that the therapy or the therapeutic agents must be administered at the same time and/or formulated for delivery together, although these methods of delivery are within the scope described herein. The therapeutic agents in the combination can be administered concurrently with, prior to, or after, one or more other additional therapies or therapeutic agents. The therapeutic agents or therapeutic protocol can be administered in any order. In general, each agent will be administered at a dose and/or on a time schedule determined for that agent. It will further be appreciated that the additional therapeutic agent utilized in this combination may be administered together or separately in different compositions. In general, it is expected that additional therapeutic agents utilized in combination be utilized at levels that do not exceed the levels at which they are utilized individually. In some embodiments, the levels utilized in combination will be lower than those utilized individually.

Complementarity Determining Region: The terms “complementarity determining region” or “CDR,” as used herein, refer to the sequences of amino acids within antibody variable regions which confer antigen specificity and binding affinity. For example, in general, there are three CDRs in each heavy chain variable region (e.g., CDR-H1, CDR-H2, and CDR-H3) and three CDRs in each light chain variable region (CDR-L1, CDR-L2, and CDR-L3). The precise amino acid sequence boundaries of a given CDR can be determined using any of a number of well-known schemes, including those described by Kabat et al., 1991, “Sequences of Proteins of Immunological Interest,” 5th Ed. Public Health Service, National Institutes of Health, Bethesda, MD (“Kabat” numbering scheme), Al-Lazikani et al., 1997, JMB 273:927-948 (“Chothia” numbering scheme) and ImMunoGenTics (IMGT) numbering (Lefranc, 1999, The Immunologist 7:132-136; Lefranc et al., 2003, Dev. Comp. Immunol. 27:55-77 (“IMGT” numbering scheme). For example, for classic formats, under Kabat, the CDR amino acid residues in the heavy chain variable domain (VH) are numbered 31-35 (CDR-H1), 50-65 (CDR-H2), and 95-102 (CDR-H3); and the CDR amino acid residues in the light chain variable domain (VL) are numbered 24-34 (CDR-L1), 50-56 (CDR-L2), and 89-97 (CDR-L3). Under Chothia, the CDR amino acids in the VH are numbered 26-32 (CDR-H1), 52-56 (CDR-H2), and 95-102 (CDR-H3); and the amino acid residues in VL are numbered 26-32 (CDR-L1), 50-52 (CDR-L2), and 91-96 (CDR-L3). By combining the CDR definitions of both Kabat and Chothia, the CDRs consist of amino acid residues 26-35 (CDR-H1), 50-65 (CDR-H2), and 95-102 (CDR-H3) in human VH and amino acid residues 24-34 (CDR-L1), 50-56 (CDR-L2), and 89-97 (CDR-L3) in human VL. Under IMGT the CDR amino acid residues in the VH are numbered approximately 26-35 (CDR-H1), 51-57 (CDR-H2) and 93-102 (CDR-H3), and the CDR amino acid residues in the VL are numbered approximately 27-32 (CDR-L1), 50-52 (CDR-L2), and 89-97 (CDR-L3) (numbering according to “Kabat”). Under IMGT, the CDR regions of an antibody can be determined using the program IMGT/DomainGap Align.

ECOG performance status score: The term “ECOG performance status score” as used herein refers to a subject's score on the Eastern Cooperative Oncology Group (ECOG) performance status score, as described in Oken et al., 1982, Am J Clin Oncol 5(6):649-55. Scores can range from 0 to 5:

| Score | Characteristics |
|---|---|
| 0 | Fully active, able to carry on all pre-disease performance |
| | without restriction |
| 1 | Restricted in physically strenuous activity but ambulatory |
| | and able to carry out work of a light or sedentary nature |
| | e.g., light housework, office work |
| 2 | Ambulatory and capable of all self-care but unable to |
| | carry out any work activities. Up and about more than |
| | 50% of waking hours |
| 3 | Capable of only limited self-care, confined to bed or |
| | chair more than 50% of waking hours |
| 4 | Completely disabled. Cannot carry on any self-care. |
| | Totally confined to bed or chair |
| 5 | Death |

Effective amount: By the term “effective amount” or “therapeutically effective amount” or “pharmaceutically effective amount”, is meant the amount or quantity of active agent (or combination of agents) that is sufficient to elicit the required or desired response, or in other words, the amount that is sufficient to elicit an appreciable biological response when administered to a subject. Said amount preferably relates to an amount that is therapeutically or in a broader sense also prophylactically effective against the progression of a disease or disorder as disclosed herein. It is understood that an “effective amount” or a “therapeutically effective amount” can vary from subject to subject, due to variation in metabolism of an agent, age, weight, general condition of the subject, the condition being treated, the severity of the condition being treated, and the judgment of the prescribing physician.

Half Antibody: The term “half antibody” refers to a molecule that comprises at least one antigen binding module (ABM) or ABM chain, wherein the ABM has the ability to bind to an antigen non-covalently, reversibly and specifically. The half antibody can associate with another molecule comprising an ABM or ABM chain through, e.g., a disulfide bridge or molecular interactions (e.g., knob-in-hole interactions between Fc heterodimers). A half antibody can be composed of one polypeptide chain or more than one polypeptide chains (e.g., the two polypeptide chains of a Fab). In an embodiment, a half-antibody comprises an Fc region.

An example of a half antibody is a molecule comprising a heavy and light chain of an antibody (e.g., an IgG antibody). Another example of a half antibody is a molecule comprising a first polypeptide comprising a VL domain and a CL domain, and a second polypeptide comprising a VH domain, a CH1 domain, a hinge domain, a CH2 domain, and a CH3 domain, where the VL and VH domains form an ABM. Yet another example of a half antibody is a polypeptide comprising an scFv domain, a CH2 domain and a CH3 domain.

A half antibody might include more than one ABM, for example a half-antibody comprising (in N- to C-terminal order) an scFv domain, a CH2 domain, a CH3 domain, and another scFv domain.

Half antibodies might also include an ABM chain that when associated with another ABM chain in another half antibody forms a complete ABM.

Single Chain Fab or scFab: The terms “single chain Fab” and “scFab” mean a polypeptide comprising an antibody heavy chain variable domain (VH), an antibody constant domain 1 (CH1), an antibody light chain variable domain (VL), an antibody light chain constant domain (CL) and a linker, such that the VH and VL are in association with one another and the CH1 and CL are in association with one another. In some embodiments, the antibody domains and the linker have one of the following orders in N-terminal to C-terminal direction: a) VH-CH1-linker-VL-CL, b) VL-CL-linker-VH-CH1, c) VH-CL-linker-VL-CH1 or d) VL-CH1-linker-VH-CL. The linker can be a polypeptide of at least 30 amino acids, for example between 32 and 50 amino acids. The single chain Fabs are stabilized via the natural disulfide bond between the CL domain and the CH1 domain.

Single Chain Fv or scFv: The term “single-chain Fv” or “scFv” as used herein refers to antibody fragments that comprise the VH and VL domains of an antibody, where these domains are present in a single polypeptide chain. The Fv polypeptide can further comprise a polypeptide linker between the VH and VL domains which enables the scFv to form the desired structure for antigen-binding. For a review of scFv see Pluckthun in The Pharmacology of Monoclonal Antibodies, vol. 113, Rosenburg and Moore eds., 1994, Springer-Verlag, New York, pp. 269-315.

Specifically (or selectively) binds: The term “specifically (or selectively) binds” to an antigen or an epitope refers to a binding reaction that is determinative of the presence of a cognate antigen or an epitope in a heterogeneous population of proteins and other biologics. An ABM typically also has a dissociation rate constant (KD) (koff/kon) of less than 5×10⁻²M, less than 10⁻²M, less than 5×10⁻³M, less than 10⁻³M, less than 5×10⁻⁴M, less than 10⁻⁴M, less than 5×10⁻⁵M, less than 10⁻⁵M, less than 5×10⁻⁶M, less than 10⁻⁶M, less than 5×10⁻⁷M, less than 10⁻⁷M, less than 5×10⁻⁸M, less than 10⁻⁸M, less than 5×10⁻⁹M, or less than 10⁻⁹M, and binds to the target antigen with an affinity that is at least two-fold greater than its affinity for binding to a non-specific antigen (e.g., HSA). Binding affinity can be measured using a Biacore, SPR or BLI assay. The term “specifically binds” does not exclude cross-species reactivity. For example, an antigen-binding module (e.g., an antigen-binding fragment of an antibody) that “specifically binds” to an antigen from one species can also “specifically bind” to that antigen in one or more other species. Thus, such cross-species reactivity does not itself alter the classification of an antigen-binding module as a “specific” binder. In certain embodiments, an antigen-binding module that specifically binds to a human antigen has cross-species reactivity with one or more non-human mammalian species, e.g., a primate species (including but not limited to one or more of Macaca fascicularis, Macaca mulatta, and Macaca nemestrina) or a rodent species, e.g., Mus musculus. In other embodiments, the antigen-binding module does not have cross-species reactivity.

Subject: The term “subject” includes human and non-human animals. Non-human animals include all vertebrates, e.g., mammals and non-mammals, such as non-human primates, sheep, dog, cow, chickens, amphibians, and reptiles. Except when noted, the terms “patient” or “subject” are used herein interchangeably.

Therapeutically effective amount: A “therapeutically effective amount” refers to an amount effective, at dosages and for periods of time necessary, to achieve a desired therapeutic result.

Treat, Treatment, Treating: As used herein, the terms “treat”, “treatment” and “treating” refer to the reduction or amelioration of the progression, severity and/or duration of a disease or disorder (e.g., a B cell malignancy), or the amelioration of the progression, severity and/or duration one or more symptoms (e.g., one or more discernible symptoms) of a disorder (e.g., CRS) resulting from the administration of one or more anti-CD19 agents. In some embodiments, the terms “treat”, “treatment” and “treating” refer to the amelioration of at least one measurable physical parameter of a disorder, such as growth of a tumor, not necessarily discernible by the patient. In other embodiments the terms “treat”, “treatment” and “treating” refer to the inhibition of the progression of a disorder, either physically by, e.g., stabilization of a discernible symptom, physiologically by, e.g., stabilization of a physical parameter, or both. In some embodiments, the terms “treat”, “treatment” and “treating” can refer to the reduction or stabilization of tumor size or cancerous cell count.

Trispecific binding molecules: The term “trispecific binding molecules” or “TBMs” refers to molecules that specifically bind to three antigens and comprise three or more antigen-binding domains. The TBMs of the disclosure comprise at least one antigen-binding domain which is specific for CD19, at least one antigen-binding domain which is specific for CD3, and at least one antigen-binding domain which is specific for CD2.

VH: The term “VH” refers to the variable region of an immunoglobulin heavy chain of an antibody, including the heavy chain of an Fv, scFv, dsFv or Fab.

VL: The term “VL” refers to the variable region of an immunoglobulin light chain, including the light chain of an Fv, scFv, dsFv or Fab.

### Anti-CD19×Anti-CD3×Anti-CD2 Trispecific Agents

Disclosed herein, at least in part, is anti-CD19×anti-CD3×anti-CD2 trispecific agent that targets CD19+ cells (malignant B cells as well as normal B cells and follicular dendritic cells), and engages CD3 (TCR signaling component) and CD2 (a costimulatory receptor) on T cells which leads to redirected T-cell cytotoxicity towards CD19-positive malignant cells.

Binding of the anti-CD19×anti-CD3×anti-CD2 trispecific agent simultaneously to multiple molecules with CD19 on malignant B cells, the CD3 subunit of the T cell receptor (TCR) complex as well as CD2 on T cells is thought to lead to TCR crosslinking and CD2 costimulation, and subsequent formation of a cytolytic immune synapse, resulting in activation of T cells and specific lysis of malignant B cells. Without being bound by theory, engaging CD2 along with CD3 is thought to provide a co-stimulatory signal to efficiently activate T cells and lead to a long-lasting T-cell response. Such a co-stimulatory signal may improve T cell responses against tumors and help to overcome limitations of CD3-bispecific antibodies.

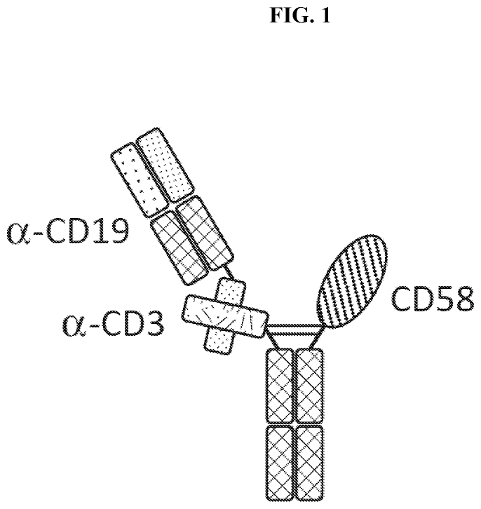

The sequences relating to the anti-CD19×anti-CD3×anti-CD2 trispecific agent are given in the table below. The anti-CD19×anti-CD3×anti-CD2 trispecific agent and methods of making this are also disclosed in PCT/US2020/033559 (WO/2020/236792) which is herein incorporated by reference. The anti-CD19×anti-CD3×anti-CD2 trispecific agent may be the CD3hi TSP1 (H variant) in Table 19C in PCT/US2020/033559 (WO/2020/236792) which is herein incorporated by reference. The first half antibody heavy chain comprises CD19 and CD3 binding sequences. The first half antibody light chain comprises CD19 binding sequences. The second half antibody comprises CD2 binding sequences (The CD2 binding sequence may be a portion of a CD58 sequence). See for example FIG. 1 to see the format of an anti-CD19×anti-CD3×anti-CD2 trispecific agent. The anti-CD2 portion is shown as a CD58 moeity.

TABLE 1
CD19 Binder Sequences

| | | | SEQ ID |
|---|---|---|---|
| Chain | Portion | Sequence | NO: |
| VH | CDR-H1 | GYTFTTYWIQ | 1 |
| | (Combined) | | |
| | CDR-H2 | AVYPGDADTRYTQKFQG | 2 |
| | (Combined) | | |
| | CDR-H3 | DAGLEYYALDY | 3 |
| | (Combined) | | |
| | CDR-H1 (Kabat) | TYWIQ | 4 |
| | CDR-H2 (Kabat) | AVYPGDADTRYTQKFQG | 5 |
| | CDR-H3 (Kabat) | DAGLEYYALDY | 6 |
| | CDR-H1 | GYTFTTY | 7 |
| | (Chothia) | | |
| | CDR-H2 | YPGDAD | 8 |
| | (Chothia) | | |
| | CDR-H3 | DAGLEYYALDY | 9 |
| | (Chothia) | | |
| | CDR-H1 (IMGT) | GYTFTTYW | 10 |
| | CDR-H2 (IMGT) | VYPGDADT | 11 |
| | CDR-H3 (IMGT) | GRDAGLEYYALDY | 12 |
| | VH | QVQLVQSGAEVKKPGAS | 13 |
| | | VKVSCKASGYTFTTYWI | |
| | | QWVRQAPGQRLEWMGAV | |
| | | YPGDADTRYTQKFQGRV | |
| | | TLTADRSASTAYMELSS | |
| | | LRSEDTAVYYCGRDAGL | |
| | | EYYALDYWGQGTLVTVS | |
| | | S | |
| VL | CDR-L1 | RASQDVGTAVA | 14 |
| | (Combined) | | |
| | CDR-L2 | WASTRHT | 15 |
| | (Combined) | | |
| | CDR-L3 | QQYANFPLYT | 16 |
| | (Combined) | | |
| | CDR-L1 (Kabat) | RASQDVGTAVA | 17 |
| | CDR-L2 (Kabat) | WASTRHT | 18 |
| | CDR-L3 (Kabat) | QQYANFPLYT | 19 |
| | CDR-L1 | SQDVGTA | 20 |
| | (Chothia) | | |
| | CDR-L2 | WAS | 21 |
| | (Chothia) | | |
| | CDR-L3 | YANFPLY | 22 |
| | (Chothia) | | |
| | CDR-L1 (IMGT) | QDVGTA | 23 |
| | CDR-L2 (IMGT) | WAS | 24 |
| | CDR-L3 (IMGT) | QQYANFPLYT | 25 |
| | VL | EIVMTQSPATLSVSPGE | 26 |
| | | RATLSCRASQDVGTAVA | |
| | | WYQQKPGQAPRLLIYWA | |
| | | STRHTGIPARFSGSGSG | |
| | | TEFTLTISSLQSEDFAV | |
| | | YFCQQYANFPLYTFGQG | |
| | | TKLEIK | |

TABLE 2
CD3 Binders - CDR sequences according to
Kabat numbering scheme

| Binding | | | SEQ ID | | SEQ ID | | SEQ ID |
|---|---|---|---|---|---|---|---|
| Domain | Chain | CDR1 | NO: | CDR2 | NO: | CDR3 | NO: |
| CD3 | VH | TYAMN | 27 | RIRSKYN | 28 | HGNFGNS | 29 |
| | | | | NYATYYA | | YVSWFAY | |
| | | | | DSVKD | | | |
| | VL | RSSTG | 30 | GTNKRAP | 31 | ALWYSNL | 32 |
| | | AVTTS | | | | WV | |
| | | NYAN | | | | | |

TABLE 3
CD3 Binders - Variable domain sequences

| | | SEQ |
|---|---|---|
| | | ID |
| Chain | Sequence | NO: |
| VH | EVQLVESGGGLVQPGGSLKLSCAASGFTFNTYAMNWVRQASGKGLEWVGR | 33 |
| | IRSKYNNYATYYADSVKDRFTISRDDSKSTLYLQMNSLKTEDTAVYYCVR | |
| | HGNFGNSYVSWFAYWGQGTLVTVSS | |
| VL | QAVVTQEPSLTVSPGGTVTLTCRSSTGAVTTSNYANWVQQKPGQAPRGLI | 34 |
| | GGTNKRAPWTPARFSGSLLGDKAALTLSGAQPEDEAEYFCALWYSNLWVF | |
| | GGGTKLTVL | |
| scFv | EVQLVESGGGLVQPGGSLKLSCAASGFTFNTYAMNWVRQASGKGLEWVGR | 35 |
| | IRSKYNNYATYYADSVKDRFTISRDDSKSTLYLQMNSLKTEDTAVYYCVR | |
| | HGNFGNSYVSWFAYWGQGTLVTVSSGGGGSGGGGSGGGGSGGGGSQAVVT | |
| | QEPSLTVSPGGTVTLTCRSSTGAVTTSNYANWVQQKPGQAPRGLIGGTNK | |
| | RAPWTPARFSGSLLGDKAALTLSGAQPEDEAEYFCALWYSNLWVFGGGTK | |
| | LTVL | |

TABLE 4
CD2 binder

| | | SEQ ID |
|---|---|---|
| | Sequence | NO: |
| CD2 | SQQIYGVVYGNVTFHVPSNVPLKEVLWKKQKD | 36 |
| binding | KVAELENSEFRAFSSFKNRVYLDTVSGSLTIY | |
| sequence | NLTSSDEDEYEMESPNITDTMKFFLYVLES | |

TABLE 5

| | | | SEQ |
|---|---|---|---|
| Construct | Chain | | ID |
| Name | Description | Amino Acid Sequence | NO: |
| CD19TSP1 | First Half | QVQLVQSGAEVKKPGASVKVSCKASGYTFTTYWIQWVRQA | 37 |
| | Antibody | PGQRLEWMGAVYPGDADTRYTQKFQGRVTLTADRSASTAY | |
| | Heavy Chain | MELSSLRSEDTAVYYCGRDAGLEYYALDYWGQGTLVTVSS | |
| | (includes Fc | ASTKGPSVFPLAPSSKSTSGGTAALGCLVKDYFPEPVTVS | |
| | sequence) | WNSGALTSGVHTFPAVLQSSGLYSLSSVVTVPSSSLGTQT | |
| | | YICNVNHKPSNTKVDKRVEPKSCGGGGSGGGGSEVQLVES | |
| | | GGGLVQPGGSLKLSCAASGFTFNTYAMNWVRQASGKGLEW | |
| | | VGRIRSKYNNYATYYADSVKDRFTISRDDSKSTLYLQMNS | |
| | | LKTEDTAVYYCVRHGNFGNSYVSWFAYWGQGTLVTVSSGG | |
| | | GGSGGGSGGGGGSGGGGSQAVVTQEPSLTVSPGGTVTLTC | |
| | | RSSTGAVTTSNYANWVQQKPGQAPRGLIGGTNKRAPWTPA | |
| | | RFSGSLLGDKAALTLSGAQPEDEAEYFCALWYSNLWVFGG | |
| | | GTKLTVLGGGGSDKTHTCPPCPAPELLGGPSVFLFPPKPK | |
| | | DTLMISRTPEVTCVVVAVSHEDPEVKFNWYVDGVEVHNAK | |
| | | TKPREEQYASTYRVVSVLTVLHQDWLNGKEYKCKVSNKAL | |
| | | AAPIEKTISKAKGQPREPQVCTLPPSREEMTKNQVSLSCA | |
| | | VKGFYPSDIAVEWESNGQPENNYKTTPPVLDSDGSFFLVS | |
| | | KLTVDKSRWQQGNVFSCSVMHEALHNHYTQKSLSLSPGK | |
| | First Half | EIVMTQSPATLSVSPGERATLSCRASQDVGTAVAWYQQKP | 38 |
| | Antibody | GQAPRLLIYWASTRHTGIPARFSGSGSGTEFTLTISSLQS | |
| | Light Chain | EDFAVYFCQQYANFPLYTFGQGTKLEIKRTVAAPSVFIFP | |
| | | PSDEQLKSGTASVVCLLNNFYPREAKVQWKVDNALQSGNS | |
| | | QESVTEQDSKDSTYSLSSTLTLSKADYEKHKVYACEVTHQ | |
| | | GLSSPVTKSFNRGEC | |
| | Second Half | SQQIYGVVYGNVTFHVPSNVPLKEVLWKKQKDKVAELENS | 39 |
| | Antibody | EFRAFSSFKNRVYLDTVSGSLTIYNLTSSDEDEYEMESPN | |
| | (includes Fc | ITDTMKFFLYVLESGGGGSDKTHTCPPCPAPELLGGPSVF | |
| | sequence) | LFPPKPKDTLMISRTPEVTCVVVAVSHEDPEVKFNWYVDG | |
| | | VEVHNAKTKPREEQYASTYRVVSVLTVLHQDWLNGKEYKC | |
| | | KVSNKALAAPIEKTISKAKGQPREPQVYTLPPCREEMTKN | |
| | | QVSLWCLVKGFYPSDIAVEWESNGQPENNYKTTPPVLDSD | |
| | | GSFFLYSKLTVDKSRWQQGNVFSCSVMHEALHNHYTQKSL | |
| | | SLSPGK | |

TABLE 6
Nucleotide sequences

| | | | SEQ |
|---|---|---|---|
| Construct | Chain | | ID |
| Name | Description | Nucleic Acid Sequence | NO: |
| CD19TSP1 | First Half | ATGCCACTGCTGCTTCTACTGCCACTCCTGTGGGCAGGA | 40 |
| | Antibody | GCACTGGCCCAAGTGCAACTGGTGCAGTCCGGTGCCGAA | |
| | Heavy Chain | GTGAAGAAGCCCGGTGCCTCTGTGAAGGTGTCCTGCAAG | |
| | (includes | GCGTCGGGATACACGTTCACCACTTACTGGATTCAGTGG | |
| | signal | GTCAGACAGGCCCCGGGACAGAGACTGGAGTGGATGGGA | |
| | peptide | GCCGTGTACCCCGGAGATGCAGACACTCGCTACACCCAG | |
| | sequence) | AAGTTCCAGGGCCGCGTGACTTTGACCGCCGACAGAAGC | |
| | | GCCAGCACCGCCTACATGGAGCTTTCATCCCTCCGGAGC | |
| | | GAGGATACTGCCGTATACTATTGCGGAAGGGATGCCGGC | |
| | | CTGGAATACTATGCCCTCGACTACTGGGGACAGGGGACC | |
| | | CTCGTGACTGTGTCCAGCGCGAGCACCAAGGGCCCCAGC | |
| | | GTGTTCCCGCTGGCCCCATCATCCAAGTCCACCTCGGGA | |
| | | GGGACTGCTGCGCTCGGTTGCCTTGTGAAGGACTACTTC | |
| | | CCCGAGCCCGTGACTGTGTCGTGGAACAGCGGGGCTCTG | |
| | | ACCAGCGGGGTTCACACCTTTCCCGCCGTGCTGCAGTCC | |
| | | TCGGGACTCTACAGCCTGTCCTCCGTGGTCACGGTCCCG | |
| | | TCGTCGTCGCTGGGGACCCAGACCTACATTTGCAACGTG | |
| | | AACCACAAACCCTCCAACACAAAAGTGGACAAAAGGGTG | |
| | | GAACCTAAGTCCTGTGGAGGGGGTGGATCAGGCGGAGGA | |
| | | GGATCGGAAGTCCAGCTCGTCGAATCAGGGGGAGGGCTT | |
| | | GTGCAACCAGGAGGCTCCCTCAAGCTGTCTTGCGCAGCG | |
| | | TCCGGTTTCACTTTCAACACTTATGCGATGAATTGGGTC | |
| | | CGCCAAGCCAGTGGGAAGGGCCTGGAGTGGGTCGGACGG | |
| | | ATCAGATCCAAGTACAACAACTACGCGACATACTACGCC | |
| | | GACTCCGTGAAGGATCGCTTCACCATCAGCCGGGATGAC | |
| | | TCCAAGAGCACCTTGTACCTCCAAATGAACAGCCTTAAG | |
| | | ACCGAGGACACTGCGGTGTACTACTGCGTGAGACACGGC | |
| | | AACTTCGGAAACTCCTACGTGTCCTGGTTCGCCTACTGG | |
| | | GGACAGGGCACCCTTGTCACTGTGTCAAGCGGAGGCGGT | |
| | | GGTTCGGGTGGCGGAGGTTCCGGAGGAGGAGGTTCGGGC | |
| | | GGTGGTGGATCACAGGCCGTCGTGACTCAGGAACCATCC | |
| | | CTGACTGTGTCCCCCGGTGGAACCGTGACCCTCACCTGT | |
| | | CGCTCCTCAACCGGAGCCGTGACCACCTCCAACTACGCT | |
| | | AATTGGGTGCAGCAGAAGCCAGGACAAGCCCCACGGGGA | |
| | | CTGATTGGGGGCACCAACAAGAGGGCTCCTTGGACCCCA | |
| | | GCCCGCTTCTCGGGCTCCCTGTTGGGCGACAAGGCCGCT | |
| | | CTGACCCTGTCCGGTGCACAGCCGGAGGATGAAGCCGAA | |
| | | TACTTCTGCGCGCTGTGGTACTCCAACCTCTGGGTGTTC | |
| | | GGCGGAGGGACCAAGCTGACTGTGTTGGGAGGAGGGGGG | |
| | | AGTGACAAGACTCACACGTGTCCGCCTTGCCCAGCACCC | |
| | | GAGCTACTGGGAGGACCGAGCGTGTTCCTGTTTCCCCCG | |
| | | AAGCCGAAGGATACCCTGATGATCTCCCGCACTCCTGAA | |
| | | GTGACTTGCGTGGTGGTGGCAGTGTCCCACGAGGACCCG | |
| | | GAAGTCAAGTTTAATTGGTACGTGGATGGCGTGGAGGTG | |
| | | CACAACGCAAAGACCAAGCCTCGCGAGGAGCAGTACGCC | |
| | | AGCACCTACCGGGTGGTGTCCGTCCTGACGGTGCTGCAC | |
| | | CAGGACTGGCTGAACGGGAAGGAGTACAAGTGCAAAGTG | |
| | | TCAAATAAGGCTTTGGCCGCCCCTATTGAGAAAACCATC | |
| | | TCAAAGGCCAAGGGCCAACCCAGGGAACCTCAAGTGTGC | |
| | | ACCCTCCCACCTTCGCGAGAAGAGATGACCAAGAACCAG | |
| | | GTGTCCCTGTCCTGCGCCGTGAAGGGCTTCTACCCCTCC | |
| | | GATATCGCCGTGGAGTGGGAATCTAACGGACAGCCGGAG | |
| | | AACAACTACAAGACCACTCCGCCGGTGCTGGACAGCGAC | |
| | | GGCTCCTTCTTCCTCGTGTCGAAACTGACCGTGGACAAG | |
| | | TCACGGTGGCAGCAGGGCAATGTGTTCAGCTGCTCAGTC | |
| | | ATGCATGAGGCCCTCCACAACCACTACACTCAGAAGTCC | |
| | | CTGTCGCTTTCCCCCGGAAAA | |
| | First Half | ATGTCGGTCCTGACCCAAGTGCTGGCCCTCCTTCTCCTG | 41 |
| | Antibody | TGGCTGACCGGGACCAGATGCGAAATCGTCATGACTCAG | |
| | Light Chain | AGCCCGGCAACCCTGTCCGTGAGCCCTGGAGAACGGGCC | |
| | (includes | ACTCTGAGCTGTCGGGCGTCACAGGACGTGGGAACTGCC | |
| | signal | GTGGCCTGGTATCAGCAGAAGCCGGGACAGGCTCCTAGG | |
| | peptide | TTGCTCATCTACTGGGCGTCCACTCGCCACACCGGAATC | |
| | sequence) | CCAGCCCGCTTCTCCGGCTCGGGTTCTGGCACCGAGTTC | |
| | | ACCCTGACCATTTCCTCCCTCCAATCCGAGGATTTCGCC | |
| | | GTGTACTTCTGCCAACAATACGCCAACTTCCCCCTGTAC | |
| | | ACATTTGGCCAGGGGACCAAGCTGGAGATTAAGCGTACG | |
| | | GTGGCCGCTCCCAGCGTGTTCATCTTCCCCCCCAGCGAC | |
| | | GAGCAGCTGAAGAGCGGCACCGCCAGCGTGGTGTGCCTG | |
| | | CTGAACAACTTCTACCCCCGGGAGGCCAAGGTGCAGTGG | |
| | | AAGGTGGACAACGCCCTGCAGAGCGGCAACAGCCAGGAG | |
| | | AGCGTCACCGAGCAGGACAGCAAGGACTCCACCTACAGC | |
| | | CTGAGCAGCACCCTGACCCTGAGCAAGGCCGACTACGAG | |
| | | AAGCATAAGGTGTACGCCTGCGAGGTGACCCACCAGGGC | |
| | | CTGTCCAGCCCCGTGACCAAGAGCTTCAACAGGGGCGAG | |
| | | TGC | |
| | Second Half | ATGCCTCTGCTGCTCCTGCTGCCTCTGCTCTGGGCCGGA | 42 |
| | Antibody | GCTTTGGCATCACAGCAAATCTACGGCGTGGTGTACGGC | |
| | (includes | AACGTGACCTTCCATGTCCCCTCCAATGTGCCGCTGAAG | |
| | signal | GAAGTGCTCTGGAAGAAGCAGAAGGACAAGGTCGCGGAA | |
| | peptide | CTGGAAAACTCCGAGTTTCGCGCCTTCTCCTCCTTCAAA | |
| | sequence) | AACCGGGTGTACCTGGACACCGTGTCCGGGAGCCTTACT | |
| | | ATCTACAACCTGACCTCCTCGGACGAGGATGAGTATGAG | |
| | | ATGGAGAGCCCAAACATTACCGACACCATGAAGTTCTTC | |
| | | CTCTACGTGCTGGAATCGGGTGGAGGCGGAAGCGATAAG | |
| | | ACTCACACGTGTCCACCTTGTCCCGCACCCGAACTCCTG | |
| | | GGGGGACCTTCCGTGTTTCTCTTCCCCCCTAAACCGAAG | |
| | | GACACCTTGATGATCTCCCGCACTCCTGAAGTGACCTGT | |
| | | GTGGTGGTGGCCGTGTCCCACGAGGACCCAGAAGTCAAG | |
| | | TTTAATTGGTACGTGGACGGAGTCGAGGTGCACAACGCG | |
| | | AAAACCAAACCGCGGGAGGAGCAGTACGCCTCCACCTAC | |
| | | CGGGTGGTGTCCGTCCTCACTGTGCTGCACCAGGACTGG | |
| | | CTCAACGGAAAGGAGTACAAGTGCAAAGTGTCCAACAAA | |
| | | GCCTTGGCGGCCCCAATCGAAAAGACGATCTCCAAGGCC | |
| | | AAGGGACAGCCGCGCGAACCTCAAGTCTACACCCTGCCT | |
| | | CCTTGCCGCGAGGAAATGACCAAGAACCAGGTGTCACTG | |
| | | TGGTGTCTGGTCAAGGGATTCTACCCTTCCGATATCGCA | |
| | | GTGGAGTGGGAAAGCAACGGCCAACCAGAGAACAACTAT | |
| | | AAGACCACACCCCCGGTGCTCGATTCCGACGGCTCATTC | |
| | | TTCCTGTACTCCAAGCTGACCGTGGACAAGTCACGGTGG | |
| | | CAGCAGGGGAACGTGTTCAGCTGCTCCGTGATGCATGAA | |
| | | GCCCTGCACAATCATTACACTCAGAAGTCCCTGTCGCTG | |
| | | AGCCCCGGAAAA | |

In some embodiments, the anti-CD19×anti-CD3×anti-CD2 trispecific agent can comprise a CD19 binding portion with a CDR-H1, a CDR-H2, and a CDR-H3 having the amino acid sequences of SEQ ID NO:1, SEQ ID NO:2, and SEQ ID NO:3, and a CDR-L1, a CDR-L2, and a CDR-L3 having the amino acid sequences of SEQ ID NO:14, SEQ ID NO:15, and SEQ ID NO:16.

In some embodiments, the anti-CD19×anti-CD3×anti-CD2 trispecific agent can comprise a CD19 binding portion with a CDR-H1, a CDR-H2, and a CDR-H3 having the amino acid sequences of SEQ ID NO:4, SEQ ID NO:5, and SEQ ID NO:6, and a CDR-L1, a CDR-L2, and a CDR-L3 having the amino acid sequences of SEQ ID NO:17, SEQ ID NO:18, and SEQ ID NO:19.

In some embodiments, the anti-CD19×anti-CD3×anti-CD2 trispecific agent can comprise a CD19 binding portion with a VH having the amino acid sequence of SEQ ID NO:13. The CD19 binding portion can also comprise a VL having the amino acid sequence of SEQ ID NO:26. The CD19 binding portion can also comprise both a VH having the amino acid sequence of SEQ ID NO:13 and a VL having the amino acid sequence of SEQ ID NO:26. In some embodiments, the anti-CD19×anti-CD3×anti-CD2 trispecific agent can comprise a CD19 binding portion which comprises any of the amino acid sequences in Table 1.

In some embodiments, the anti-CD19×anti-CD3×anti-CD2 trispecific agent can comprise a CD3 binding portion with a CDR-H1, a CDR-H2, and a CDR-H3 having the amino acid sequences of SEQ ID NO:27, SEQ ID NO:28, and SEQ ID NO:29, and a CDR-L1, a CDR-L2, and a CDR-L3 having the amino acid sequences of SEQ ID NO:30, SEQ ID NO:31, and SEQ ID NO:32.

In some embodiments, the anti-CD19×anti-CD3×anti-CD2 trispecific agent can comprise a CD3 binding portion which comprises a VH having the amino acid sequence of SEQ ID NO: 33. In some embodiments, the anti-CD19×anti-CD3×anti-CD2 trispecific agent can comprise a CD3 binding portion which comprises a VL having the amino acid sequence of SEQ ID NO: 34. In some embodiments, the anti-CD19×anti-CD3×anti-CD2 trispecific agent can comprise a CD3 binding portion which comprises the amino acid sequence of SEQ ID NO: 35. In some embodiments, the anti-CD19×anti-CD3×anti-CD2 trispecific agent can comprise a CD3 binding portion which comprises any of the amino acid sequences given in Table 2 and/or Table 3.

In some embodiments the CD2 binding portion domain comprises the CD2-interacting IgV domain of its natural ligand CD58 (CD58-IgV, also referred to as anti-CD2). In some embodiments the anti-CD19×anti-CD3×anti-CD2 trispecific agent can comprise a CD2 binding portion which is a CD58 moiety. The CD58 moiety can comprise amino acid residues 30-123 of full length wild type CD58. In some embodiments, the anti-CD19×anti-CD3×anti-CD2 trispecific agent can comprise a CD2 binding portion having an amino acid sequence of SEQ ID NO: 36. In some embodiments, the anti-CD19×anti-CD3×anti-CD2 trispecific agent can comprise a CD2 binding portion which comprises any of the amino acid sequences given in Table 4.

### B Cell Malignancies

The CD19 protein is ubiquitously expressed in the B lymphocyte lineage. CD19 expression is maintained in B-lineage cells that have undergone a neoplastic transformation to ALL and B-NHL (Scheumermann and Racila 1995).

In some embodiments, the B cell malignancy is B cell acute lymphocytic leukemia (also known as B cell acute lymphoblastic leukaemia or B cell acute lymphoid leukemia) (ALL or B-ALL), e.g., relapsed and/or refractory B-ALL.

In some embodiments the invention relates to an anti-CD19×anti-CD3×anti-CD2 trispecific agent for use in treating relapsed and/or refractory B-NHL. In some embodiments the invention relates to a method of treating a subject having a relapsed and/or refractory LBCL by administering an anti-CD19×anti-CD3×anti-CD2 trispecific agent to a subject in need thereof, wherein the subject previously received CAR-T therapy. In some embodiments the invention relates to a method of treating a subject having a relapsed and/or refractory LBCL by administering an anti-CD19×anti-CD3×anti-CD2 trispecific agent to a subject in need thereof, wherein the subject did not previously receive CAR-T therapy. In some embodiments the invention relates to an anti-CD19×anti-CD3×anti-CD2 trispecific agent for use in treating relapsed and/or refractory B-ALL.

In some embodiments, the B cell malignancy is chronic lymphocytic leukemia (CLL)/small lymphocytic lymphoma (SLL), e.g., relapsed and/or refractory CLL/SLL.

In some embodiments, the B cell malignancy is marginal zone lymphoma (MZL).

In some embodiments, the B cell malignancy is extranodal marginal zone lymphoma (EMZL).

In some embodiments, the B cell malignancy is nodal marginal zone B-cell lymphoma (NZML).

In some embodiments, the B cell malignancy is splenic marginal zone B-cell lymphoma (SMZL).

Refractory or relapsed CD19-positive B-ALL may include at least 1 of the following:
i) after at least 2 or more lines of systemic therapy
ii) Relapsed or Refractory at any time after first salvage therapy or refractory relapse.
iii) Relapse at any time after hematopoietic stem cell transplant (HSCT)
iv) Refractory to, or intolerant to, or ineligible/unable to receive SOC therapeutic options including blinatumomab and inotuzumab
v) Patients with R/R B-ALL Ph+ disease and that are intolerant or refractory to available tyrosine kinase inhibitors (TKIs)

The end points and/or treatment may be measured in terms of one or more of the following criteria
1) Incidence and severity of Dose Limiting Toxicities (DLTs) (e.g. after 28 days or 35 days, depending on the dosing schedule)
2) Incidence and severity of Adverse Events (AEs) and Serious Adverse Events (SAEs) (e.g. after 21 months)
3) Frequency of dose interruptions (e.g. after 21 months)
4) Frequency of dose reductions (e.g. after 21 months)
5) Dose intensities (e.g. after 21 months)

In addition the end points and/or treatment may be measured in terms of one or more of the following criteria
1) Overall Response Rate (ORR) (e.g. after 21 months)
2) Complete Response (CR) rate (e.g. after 21 months)
3) Best Overall Response (BOR) (e.g. after 21 months)
4) Duration Of Response (DOR) (e.g. after 21 months)
5) Overall Survival (OS) (e.g. after 33 months)
6) Progression Free Survival (PFS) (e.g. after 21 months)
7) Event-free survival (EFS) (e.g. after 21 months)
8) Maximum concentration of CD19TSP1 (Cmax) (e.g. after 21 months)
9) Area Under the Curve of CD19TSP1 (AUC) (e.g. after 21 months)
10) Trough concentration of CD19TSP1 (C trough) (e.g. after 21 months)
11) Prevalence of Anti-drug antibodies (ADA) (e.g. at baseline)
12) Incidence of Anti-drug antibodies (ADA) on treatment (e.g. after 21 months)

The treatment may improve one or more of the following criteria
i) Remission rate
ii) Duration of remission
iii) Overall survival
in combination with an acceptable safety profile.

In some embodiments one or more of the following agents selected from the group consisting of tocilizumab, siltuximab, cyclophosphamide, anti-thymocyte globulin (ATG), alemtuzumab, anakinra, steroids, anti-IL-6, anti-TNF and anti-IL-1R antibodies, antihistamine, steroids (including corticosteroids), canakinumab may be administered after the anti-CD19×anti-CD3×anti-CD2 trispecific agent. For example the steroid may be prednisone or dexamethasone. In some embodiments the administration of the anti-CD19×anti-CD3×anti-CD2 trispecific agent may occur after or concurrently with acetaminophen and/or diphenhydramine. In some embodiments the administration of the anti-CD19×anti-CD3×anti-CD2 trispecific agent may occur before, after or concurrently with CRS therapy.

### Pharmaceutical Compositions and Combination Administration

The anti-CD19×anti-CD3×anti-CD2 trispecific agent can be formulated as pharmaceutical compositions containing one or more pharmaceutically acceptable excipients or carriers. To prepare pharmaceutical or sterile compositions, an anti-CD19×anti-CD3×anti-CD2 trispecific agent preparation can be combined with one or more pharmaceutically acceptable excipients and/or carriers. The compositions may be in a variety of forms. These include, for example, liquid, semi-solid and solid dosage forms, such as liquid solutions (e.g., injectable and infusible solutions), dispersions or suspensions, liposomes and suppositories. The preferred form depends on the intended mode of administration and therapeutic application. Typical preferred compositions are in the form of injectable or infusible solutions. The preferred mode of administration is parenteral (e.g., intravenous, subcutaneous, intraperitoneal, intramuscular). In a preferred embodiment, the anti-CD19×anti-CD3×anti-CD2 trispecific agent is administered by intravenous infusion or injection. In another preferred embodiment, the anti-CD19×anti-CD3×anti-CD2 trispecific agent is administered by subcutaneous injection. Therapeutic compositions typically should be sterile and stable under the conditions of manufacture and storage. The composition can be formulated as a solution, microemulsion, dispersion, liposome, or other ordered structure suitable to high antibody concentration. Sterile injectable solutions can be prepared by incorporating the active compound (i.e., antibody or antibody portion) in the required amount in an appropriate solvent, followed by filtered sterilization. Pharmaceutical compositions for use in the disclosed methods may be manufactured in conventional manner. The use of antibodies as the active ingredient of pharmaceuticals is now widespread, including the products Herceptin® (trastuzumab), Rituxan® (rituximab), Synagis® (palivizumab), etc. Techniques for lyophilisation, preparation of aqueous formulations, and purification of antibodies to a pharmaceutical grade are well known in the art. Antibodies are typically formulated either in aqueous form ready for parenteral administration or as lyophilisates for reconstitution with a suitable diluent prior to administration. In some embodiments of the disclosed methods and uses, the antibodies of the present invention are formulated as a lyophilisate. Suitable lyophilisate formulations can be reconstituted in a small liquid volume (e.g., 2 ml or less) to allow subcutaneous administration and can provide solutions with low levels of antibody aggregation. For immediate administration it is dissolved in a suitable aqueous carrier, for example sterile water for injection or sterile buffered physiological saline. If it is considered desirable to make up a solution of larger volume for administration by infusion rather than a bolus injection, may be advantageous to incorporate human serum albumin or the patient's own heparinised blood into the saline at the time of formulation. The presence of an excess of such physiologically inert protein prevents loss of antibody by adsorption onto the walls of the container and tubing used with the infusion solution. If albumin is used, a suitable concentration is from 0.5 to 4.5% by weight of the saline solution.

### Administration Methods and Rates

The trispecific agent disclosed herein can be administered by a variety of methods known in the art, although for many therapeutic applications, the preferred route/mode of administration is intravenous injection or infusion. See for example, Sachs et al., Optimal Dosing for Targeted Therapies in Oncology: Drug Development Cases Leading by Example, Clin. Cancer Res; 22(6) 2016; Bai et al, A Guide to Rational Dosing of Monoclonal Antibodies, Clin. Pharmacokinet. 2012: 51 (2) 119-135; Le Tourneau, J., Dose Escalation Methods in Phase I Cancer Clinical Trials, J Natl Cancer Inst 2009; 101:708-720; Wang, D. et al., Fixed Dosing Versus Body Size-Based Dosing of Monoclonal Antibodies in Adult Clinical Trials, J Clin Pharmacol 2009; 29:1012-1024; Hempel, G. et ano, Flat-Fixed Dosing Versus Body Surface Aread-Based Dosing of Anticancer Drugs: There Is a Difference, The Oncologist 2007: 12:924-926, Mathijssen, R., Flat-Fixed Dosing Versus Body Surface Area-Based Dosing of Anticancer Drugs in Adults: Does It Make a Difference?, The Oncologist, 2007; 12:913-923; Leveque, Evaluation of Fixed Dosing of New Anticancer Agents in Phase I Studies, Anticancer Research 28:300275-2078 (2008), Gurney, How to calculate the dose of chemotherapy, British Journal of Cancer (2002) 86, 1297-1302; For example, the antibody molecules can be administered by intravenous infusion at a rate of more than 20 mg/min, e.g., 20-40 mg/min, and typically greater than or equal to 40 mg/min to reach a dose of about 35 to 440 mg/m2, typically about 70 to 310 mg/m2, and more typically, about 110 to 130 mg/m2. In embodiments, antibody molecules can be administered by intravenous infusion at a rate of less than 10 mg/min; preferably less than or equal to 5 mg/min to reach a dose of about 1 to 100 mg/m 2, preferably about 5 to 50 mg/m2, about 7 to 25 mg/m2 and more preferably, about 10 mg/m2. The route and/or mode of administration will vary depending upon the desired results.

### Dosage Regimes

Dosage regimens are adjusted to provide the optimum desired response (e.g., a therapeutic response). For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the subjects to be treated; each unit contains a predetermined quantity of active compound calculated to produce the desired therapeutic effect in association with the required pharmaceutical carrier. The specification for the dosage unit forms of the invention are dictated by and directly dependent on (a) the unique characteristics of the active compound and the particular therapeutic effect to be achieved, and (b) the limitations inherent in the art of compounding such an active compound for the treatment of sensitivity in individuals.

### 5. ADDITIONAL EMBODIMENTS

All publications, patent applications, patents, and other references mentioned herein are incorporated by reference in their entirety.

Other features, objects, and advantages of the invention will be apparent from the description and drawings, and from the claims.

### 6. ADDITIONAL EMBODIMENTS

### Example 1

In vitro RTCC assays were developed to demonstrate CD19TSP1-mediated specific lysis of a CD19+ B cell lymphoma cell line, Karpas422. The Karpas422 cells were engineered to overexpress the firefly luciferase gene. Tumor cell killing, T cell proliferation and cytokine secretion were assessed following the co-culture of Karpas422 cells with T cells in the presence of CD19TSP1 or the isotype control antibody TSP1C (which has an anti-chicken lysozyme portion instead of CD19 binding portion i.e. anti-chicken lysoszymexCD2×CD3 targeting antibody). Luciferase signal reduction was then measured as an indicator of Karpas422 cell lysis. In addition, T cell activation was assessed by quantifying the levels of interferon gamma (IFNγ), interleukin-2 (IL-2) and tumor necrosis factor alpha (TNFα) cytokine production in the culture supernatant, whereas T cell proliferation was determined using T cell counts from flow cytometry.

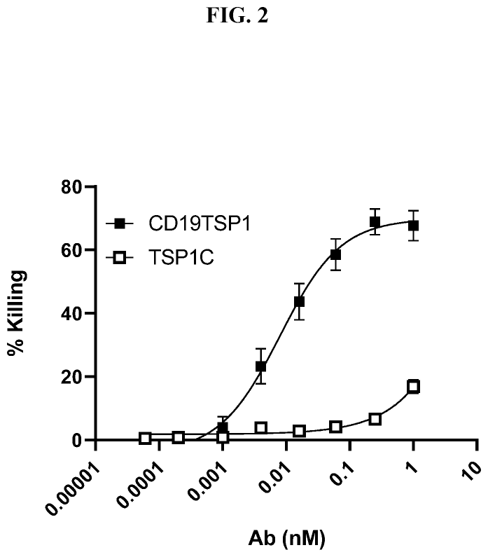

T cells from healthy donors were co-cultured with luciferase expressing Karpas422 cells at a 1:1 ratio for 96 hours in the presence of CD19TSP1 or TSP1C. % Killing (% lysis of Karpas 422 cells) was determined by the reduction in luciferase activity compared to Karpas422 cells alone. Mean values+/−SEM were plotted from twelve biological replicates (from four individual healthy donor T cells, each repeated in three independent experiments). See FIG. 2.

CD19TSP1 at doses above 0.01 nM mediated efficient killing of Karpas 422 cells by T cells from healthy human donors in a dose-dependent manner, while the isotype control antibody (TSP1C) only demonstrated negligible killing at 1 nM, the top concentration tested.

T cell proliferation was quantified using flow cytometry.

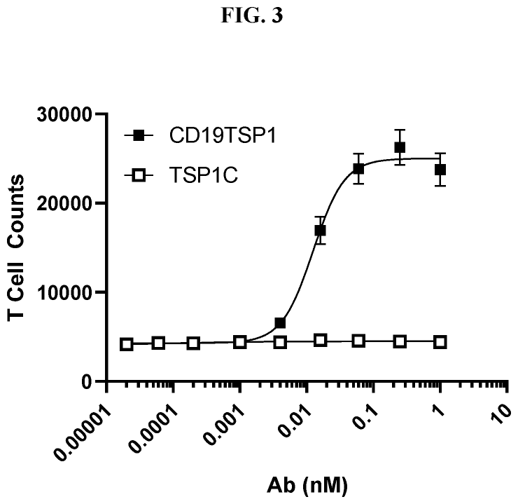

CTV-stained T cells from healthy donors were co-cultured with luciferase expressing Karpas422 cells at a 1:1 ratio for 96 hours in the presence of CD19TSP1 or TSP1C at the indicated concentrations. T cell counts were determined by gating on CTV stained T cells using flow cytometry. Mean values+/−SEM were plotted from twelve biological replicates (from four individual healthy donor T cells, each repeated in three independent experiments). See FIG. 3.

CD19TSP1 induced concentration dependent T cell proliferation in the presence of Karpas 422 cells. As expected, T cells in the TSP1C control showed no proliferation.

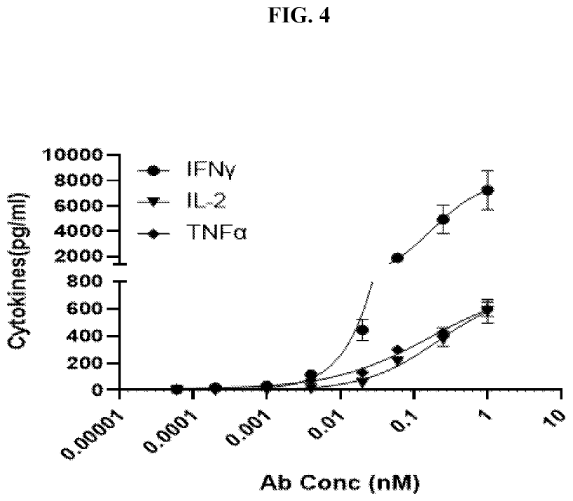

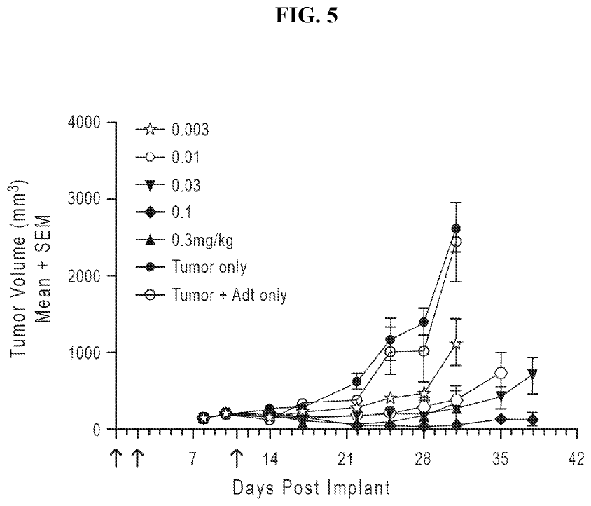

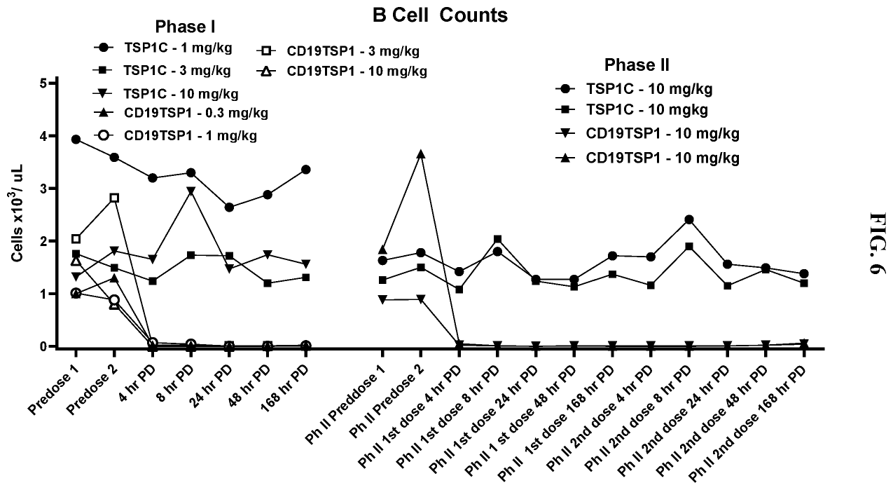

CD19TSP1 treatment results in secretion of IFNγ, IL-2 and TNFα from Karpas422-T cell co-culture. Cell culture supernatants were collected from RTCC assay 48 hrs after initiation of Karpas422-T cell co-culture. Levels of secreted IFNγ, IL-2 and TNFα were quantitated using a Custom 3-Plex MSD VPLEX immunoassay. Mean values+/−SEM were plotted from twelve biological replicates (from four individual healthy donor T cells, each repeated in three independent experiments). See FIG. 4.

Levels of IFNγ, IL-2 and TNFα in the conditioned medium from RTCC assay were quantified using a Meso Scale Discovery (MSD) VPLEX multiplex immunoassay. CD19TSP1 treatment of Karpas422 and T cell co-culture resulted in increased secretion of IFNγ, IL-2 and TNFα, whereas TSP1C induced no to low levels of secretion (data not shown). The magnitude of secreted IFNγ was significantly higher than IL-2 and TNFα.

### Example 2

Two preclinical in vivo models were used to evaluate the activity of CD19TSP1: an AdT model and a hCD34+ model. The AdT model was used to evaluate the single dose activity of CD19TSP1 across a dose range against an established DLBCL tumor. The hCD34+ model was used in a multi-dose study of CD19TSP1 against an established DLBCL tumor. In both model systems, CD19TSP1 demonstrated a robust and durable anti-tumor response against the same established tumor.

CD19TSP1 showed dose dependent anti-tumor activity and was well tolerated in the AdT model with DLBCL xenograft: 5×106 OCILY19 cells were implanted subcutaneously (s.c.) on Day 0. Two days later, 15 million PBMCs from frozen donor stock were engrafted via IV administration. 11 days post tumor implant, tumor volume reached ˜200 mm3, and animals were randomized into their respective treatment groups and received a single dose of their respective treatment. Anti-tumor activity of CD19TSP1 was assessed at different dose levels. Tumor only group (in mice without human PBMC adoptive transfer) and tumor in AdT mice (no Ab) were included as control. CD19TSP1 dosed at 0.3 mg/kg, 0.1 mg/kg and 0.03 mg/kg showed anti-tumor activity that is statistically different from the control group (p<0.05, one-way ANOVA with post-hoc Dunnetts multiple comparison). The number of CR, PR and NR are as follows for each group 0.3 mg/kg (7CR/0PR/1 NR), 0.1 mg/kg (2CR/6PR/0NR) and 0.03 mg/kg (2CR/5PR/1NR), 0.01 mg/kg (1CR/3PR/4NR) and 0.003 mg/kg (0CR/7PR/1NR). CR=complete regression; PR=partial regression; NR=no response.

CD19TSP1 showed dose dependent anti-tumor activity and durable responses against the DLBCL xenograft in the hCD34+ model: 5×10⁶ OCILY19 cells were implanted subcutaneously (s.c.). 11 days post engraftment, animals were randomized based on tumor size and donor into respective treatment groups as follows, no treatment, 0.3 mg/kg CD19TSP1 or 0.1 mg/kg CD19TSP1. Animals were treated once weekly for 3 weeks via IV dose (QWx3, IV). CD19TSP1 demonstrated dose dependent activity. Compared to the control, both 0.3 mg/kg and 0.1 mg/kg CD19TSP1 showed statistically significant anti-tumor activity (p<0.05, one-way ANOVA with post-hoc SIDAK multiple comparison). CD19TSP1 dosed at 0.3 mg/kg demonstrated robust activity with 5/8CR and 1 PR whereas the 0.1 mg/kg resulted in growth delay and was less active with 1CR and 1PR. Both the 0.1 mg/kg and 0.3 mg/kg conferred a survival benefit over untreated. CR=complete regression; PR=partial regression.

### Example 3

Evaluation of CD19TSP1 in a dose range finding toxicity study in cynomologus monkeys demonstrated that single and repeated (2 doses given one week apart) intravenous administration of CD19TSP1 was tolerated. Total B cell counts in monkeys treated with single dose (phase 1) or two doses separated by one week (phase II) of CD19TSP1 or the trispecific isotype control TSP1C (which has an anti-chicken lysozyme portion instead of CD19 binding portion i.e. anti-chicken lysoszymexCD2×CD3 targeting antibody) were measured in peripheral blood. Intravenous administration of CD19TSP1 induced sustained B cell depletion in peripheral blood (see FIG. 6) and lymphoid organs (based on histopathology evaluation).

### Example 4

CD19TSP1 was also tolerated following subcutaneous administration of a single dose (0.3 mg/kg) to cynomolgus monkeys.

### Example 5

Disclosed herein is a phase I study to characterise the safety and tolerability of an anti-CD19×anti-CD3×anti-CD2 trispecific agent (CD19TSP1), as well as to identify the maximum tolerated dose and/or recommended dose, schedule and route of administration in R/R B-cell NHL and/or R/R B-ALL. The study comprises a dose escalation part of an anti-CD19×anti-CD3×anti-CD2 trispecific agent (CD19TSP1) in R/R B-cell NHL and/or R/R B-ALL and a dose expansion part in R/R LBCL who i) received CAR-T therapy or ii) not and iii) R/R B-ALL. During the dose escalation, the safety (including the dose-dose limiting toxicity (DLT) relationship) and tolerability of an anti-CD19×anti-CD3×anti-CD2 trispecific agent (CD19TSP1) will be assessed, and schedule(s), route of administration(s) and dose(s) will be identified for use in the expansion part based on the review of these data. The recommended dose will also be guided by the available information on pharmacokinetics (PK), pharmacodynamics (PD), and preliminary anti-tumor activity. The dose escalation will be guided by an adaptive Bayesian logistic regression model (BLRM) following the Escalation with Overdose Control (EWOC) principle. Different schedule (once weekly (Q1W) or once every two weeks (Q2W) with and without priming dose) and routes of administrations (intravenous (i.v.) or subcutaneous (s.c.)) will be explored in the dose escalation groups.

An anti-CD19×anti-CD3×anti-CD2 trispecific agent (CD19TSP1) will be used in adult NHL patients for whom two or more lines of chemotherapy have failed and either having progressed (or relapsed) after autologous hematopoietic stem cell transplantation (HSCT), or being ineligible for or not consenting to the procedure.

An anti-CD19×anti-CD3×anti-CD2 trispecific agent (CD19TSP1) will be used in adult R/R ALL patients.

An anti-CD19×anti-CD3×anti-CD2 trispecific agent (CD19TSP1) will be used in adult R/R LBCL (DLBCL, double/triple hit HGBCL, PMBCL, FL3B patients who did not receive CD19 directed CAR-T therapy).

An anti-CD19×anti-CD3×anti-CD2 trispecific agent (CD19TSP1) will be used in adult R/R LBCL (DLBCL, double/triple hit HGBCL, PMBCL, FL3B) patients who received CD19 directed CAR-T therapy.

An anti-CD19×anti-CD3×anti-CD2 trispecific agent (CD19TSP1) will be used in adult R/R ALL patients.

The incidence and severity of Dose Limiting toxicities will be used as an assessment of safety of study drug. A does limiting toxicity is defined as an adverse event or abnormal laboratory value of CTCAE grade 3 or higher that occurs within the DLT evaluation period (28 days of treatment initial schedule or 35 days for priming dose schedule) and that is not primarily related to disease, disease progression, intercurrent illness, or concomitant medications.

The overall response rate (ORR), Complete Response (CR) rate, Best Overall Response (BOR), Duration of Response (DOR), Overall Survival (OS), Progression Free Survival (PFS), Event free Survival (EFS) for NHL will be based on according to Lugano response Criteria Classification criteria and for ALL on National Comprehensive Cancer Network (NCCN) guidelines as relevant.

### INCORPORATION BY REFERENCE

All publications, patents, and Accession numbers mentioned herein are hereby incorporated by reference in their entirety as if each individual publication or patent was specifically and individually indicated to be incorporated by reference.

### EQUIVALENTS

While specific embodiments of the subject invention have been discussed, the above specification is illustrative and not restrictive. Many variations of the invention will become apparent to those skilled in the art upon review of this specification and the claims below. The full scope of the invention should be determined by reference to the claims, along with their full scope of equivalents, and the specification, along with such variations.

1. A method of treating a subject having a condition which is Non-Hodgkin Lymphoma (NHL) or Acute Lymphoblastic Leukemia (ALL) by administering a therapeutically effective amount of an anti-CD19×anti-CD3×anti-CD2 trispecific agent to a subject in need thereof.

2. An anti-CD19×anti-CD3×anti-CD2 trispecific agent for use in treating a condition which is Non-Hodgkin Lymphoma (NHL) or Acute Lymphoblastic Leukemia (ALL).

3. The method of claim 1, wherein the anti-CD19×anti-CD3×anti-CD2 trispecific agent comprises (a) a first polypeptide whose amino acid sequence comprises the amino acid sequence of SEQ ID NO:37; (b) a second polypeptide whose amino acid sequence comprises the amino acid sequence of SEQ ID NO:38; and (c) a third polypeptide whose amino acid sequence comprises the amino acid sequence of SEQ ID NO:39.

4. The method of claim 1, wherein the condition is selected from the group consisting of LBCL, DLBCL, HGBCL, PMBCL, FL, FL3B, MCL, SLL, MZL and ALL.

5. The method of claim 1, wherein the condition is selected from the group consisting of DLBCL, HGBCL, PMBCL, FL3B, MCL, SLL and MZL.

6. The method of claim 1, wherein the condition is R/R DLBCL.

7. The method of claim 1, wherein the condition R/R HGBCL.

8. The method of claim 1, wherein the treatment may be after previous CAR-T therapy and/or after treatment with a CD20 monoclonal antibody containing chemotherapy regimen and/or prior autologous hematopoietic stem cell transplantation (HSCT).

9. The method of claim 1, wherein anti-CD19×anti-CD3×anti-CD2 trispecific agent may be administered Q1W or Q2W.

10. The method of claim 1, wherein the anti-CD19×anti-CD3×anti-CD2 trispecific agent may be administered intravenously or subcutaneously.

11. The method of claim 1, wherein the anti-CD19×anti-CD3×anti-CD2 trispecific agent may be administered via an initial priming dose, followed by a main dose.

12. The method of claim 1, wherein the anti-CD19×anti-CD3×anti-CD2 trispecific agent may be administered at a dose selected from the group consisting of 0.1, 0.3, 1, 3, 10, 20, 40, 80, 160, 320, 640, 1280, 2560 micrograms/kilogram (μg/kg).

13. The method of claim 1, wherein the anti-CD19×anti-CD3×anti-CD2 trispecific agent may be administered before, after or concurrently with a CRS therapy.

14. The method of claim 1, wherein anti-CD19×anti-CD3×anti-CD2 trispecific agent may be administered before, after or concurrently with tocilizumab.

15. A method of treating a subject with a CD19 associated disease or disorder which comprises administering an anti-CD19×anti-CD3×anti-CD2 trispecific agent at a dose selected from the group consisting of 0.1, 0.3, 1, 3, 10, 20, 40, 80, 160, 320, 640, 1280, 2560 micrograms/kilogram (μg/kg).

16. An anti-CD19×anti-CD3×anti-CD2 trispecific agent for use as a medicament, wherein the anti-CD19×anti-CD3×anti-CD2 trispecific agent is administered at a dose selected from the group consisting of 0.1, 0.3, 1, 3, 10, 20, 40, 80, 160, 320, 640, 1280, 2560 micrograms/kilogram (μg/kg).

17. The method of claim 15, wherein the anti-CD19×anti-CD3×anti-CD2 trispecific agent can comprise a CD19 binding portion with a CDR-H1, a CDR-H2, and a CDR-H3 having the amino acid sequences of SEQ ID NO:4, SEQ ID NO:5, and SEQ ID NO:6, and a CDR-L1, a CDR-L2, and a CDR-L3 having the amino acid sequences of SEQ ID NO:17, SEQ ID NO:18, and SEQ ID NO:19.

18. The method of claim 15, wherein the anti-CD19×anti-CD3×anti-CD2 trispecific agent comprises (a) a first polypeptide whose amino acid sequence comprises the amino acid sequence of SEQ ID NO:37; (b) a second polypeptide whose amino acid sequence comprises the amino acid sequence of SEQ ID NO:38; and (c) a third polypeptide whose amino acid sequence comprises the amino acid sequence of SEQ ID NO:39.

19. The method of claim 15, wherein the condition or CD19 associated disease or disorder is LBCL or FL3B, optionally relapsed and/or refractory LBCL or FL3B.

20. The method of claim 15, wherein the disease or disorder is systemic lupus erythematosus (SLE).

21. The method of claim 15, wherein anti-CD19×anti-CD3×anti-CD2 trispecific agent may be administered Q1W or Q2W.

22. The method of claim 15, wherein the anti-CD19×anti-CD3×anti-CD2 trispecific agent may be administered intravenously or subcutaneously.

23. The method of claim 15, wherein the anti-CD19×anti-CD3×anti-CD2 trispecific agent may be administered via an initial priming dose, followed by a main dose.

24. The method of claim 1, wherein the NHL or ALL is relapsed and/or refractory NHL or relapsed and/or refractory ALL.

25. The method of claim 6, wherein the R/R DLBCL is de novo or transformed R/R DLBCL.

26. The method of claim 7, wherein the R/R HGBCL is relapsed and/or refractory double/triple hit High-grade B-cell lymphoma (HGBCL).
